(19) United States
(12) Patent Application Publication
(10) Pub. No.: US 2025/0243469 A1
(43) Pub. Date: Jul. 31, 2025

(54) OXYGEN CARRIERS FOR MAINTAINING ORGAN VIABILITY DURING NORMOTHERMIC PERFUSION

(71) Applicant: Ohio State Innovation Foundation, Columbus, OH (US)

(72) Inventors: Andre PALMER, Westerville, OH (US); Clayton CUDDINGTON, Houston, TX (US); Bryan WHITSON, Westerville, OH (US); Sylvester BLACK, Columbus, OH (US)

(21) Appl. No.: 18/854,684

(22) PCT Filed: Apr. 6, 2023
(86) PCT No.: PCT/US2023/017765

§ 371 Date: Oct. 7, 2024

Related U.S. Application Data
(60) Provisional application No. 63/327,983, filed on Apr. 6, 2022.

(51) Int. Cl. C12N 5/00; C07K 14/805

(52) U.S. Cl. CPC C12N 5/526; C07K 14/805

(57) ABSTRACT

Described herein is a method of preserving a biological tissue sample ex vivo, the method comprising comprises contacting the tissue sample ex vivo with a perfusion solution comprising polymerized hemoglobin, wherein the perfusion solution comprises less than 5% by weight low molecular weight hemoglobin species, based on the total weight of the perfusion solution.

### CROSS-REFERENCE TO RELATED APPLICATIONS

This application claims benefit of U.S. Provisional Application No. 63/327,983, filed Apr. 6, 2022, which is hereby incorporated herein by reference in its entirety.

### STATEMENT REGARDING FEDERALLY SPONSORED RESEARCH OR DEVELOPMENT

This invention was made with government support under Grant No. W81XWH-18-1-0059 awarded by the Department of Defense, and Grant Nos. R01HL 126945, R01HL138116, R01HL156526, and R01EB021926 awarded by the National Institutes of Health. The government has certain rights in the invention.

### BACKGROUND

In the United States in 2020, there were almost 108,000 patients on the organ transplant waiting list, yet only 39,000 transplants were performed leading to 11,000 patients being removed from the waiting list due to death or sickness. A primary reason for the disparity between the number of patients on the waiting list and the number of transplants performed is an overall limited number of available organs and additionally some of the available supply of organs are deemed of poor quality and are not viable for transplantation. For organs such as the lungs, more than 80% of donor allografts offered by organ donors are declined due to poor organ quality. It is obvious that the best way to decrease the number of patients on the organ waiting list is to make more organs available for transplantation. In an effort to expand the donor pool, organs from marginal or extended criteria donors (ECD) may be evaluated. However, ECD grafts are associated with a significantly higher risk of ischemia-reperfusion injury (IRI) which leads to primary graft dysfunction and subsequently reduces organ viability. Due to a rising demand in the need for organ transplantation and a critical donor organ shortage, the need to fill this gap has increased the use of ECD and donation after cardiac death (DCD) organs viable for transplantation to lower the mortality of patients on the organ waiting list.

### SUMMARY

In accordance with the purposes of the disclosed materials and methods, as embodied and broadly described herein, the disclosed subject matter, in one aspect, relates to preserving and assessing viability of biological tissue samples.

Thus, in one aspect, a method of preserving a biological tissue sample is provided including contacting the tissue sample ex vivo with a perfusion solution comprising polymerized hemoglobin, wherein the perfusion solution comprises less than 5% by weight low molecular weight hemoglobin species, based on the total weight of the perfusion solution.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

The accompanying drawings, which are incorporated in and constitute a part of this specification, illustrate certain examples of the present disclosure and together with the description, serve to explain, without limitation, the principles of the disclosure. The application includes reference to the accompanying figures, in which:

FIG. 1 shows an exemplary NEVLP circuit is shown, wherein a sweep gas across the oxygenator is composed of 8%/6%/86% CO₂/O₂/N₂. A heat exchanger maintains the perfusate reservoir and flow path at 37° C.

FIG. 2 is an exemplary graph of the concentration of PolyhHb in the HBOC perfusate and the corresponding hematocrit of RBCs in the perfusate during NEVLP. The hematocrit decreases as a function of time for RBCs, which signifies hemolysis occurring during NEVLP. The hematocrit equivalent for PolyhHb is calculated by multiplying the concentration in g/dl by 3.

FIG. 3 is an exemplary graph of the percent change in metHb level of the synthesized PolyhHb. In this embodiment, the percent change in metHb level is lower and more controlled than the empty circuit HBOC NMP embodiments.

FIG. 4 is an exemplary average SEC-HPLC chromatogram for the PolyhHb perfusate both before and after perfusion. The size distributions overlapping each other demonstrate that the PolyhHb remains structurally intact during NEVLP.

FIG. 5 is an exemplary graph of pO₂ of the perfusate exiting the lung. PolyhHb exhibited higher post-lung pO₂s compared to RBCs after 30 minutes and compared to both perfusates by 90 minutes. The PolyhHb perfusate was also the only one to not significantly decrease in oxygenation over time.

FIG. 6 is an exemplary graph of the amount of O₂ delivered to the perfusate by the lung. RBCs exhibited lower oxygenation capacity compared to both other perfusates after 30 minutes. The RBC perfusate was also the only one to decrease in the amount of oxygen delivered over time.

FIG. 7 is an exemplary graph of the change in partial pressure of CO₂ (pCO₂) of the perfusate across the lung. Only the RBC perfusate did not have a decrease in CO₂ clearance.

FIG. 8 is an exemplary graph of PA pressure over the course of NEVLP. PA pressure for the RBC perfusates was significantly higher than all other groups across all time points. In these embodiments, three of the six RBC perfusions had to be cut short after 60 minutes due to the development of PA pressures over 100 cm H₂O.

FIG. 9 is an exemplary graph of PVR over time, which in this embodiment, shows a similar trend to PA pressure where the RBC perfusate yielded higher values compared to the PolyhHb or control perfusate.

FIG. 10 is an exemplary graph of the change in lung weight over time. In this embodiment at 60 minutes, the value for the RBC perfusates spiked due to three lungs that were then deemed non-viable and removed from the circuit. While the PolyhHb and control perfusates seem to stabilize, the RBC perfusates demonstrate a continued increase in lung weight, signifying continuing tissue damage and edema.

FIG. 11 is an exemplary graph of the change in LDH level of the various perfusates during perfusion. The PolyhHb perfusate was the only group to not achieve a significantly higher LDH release compared to the 30 minute timepoint. It also demonstrated significantly less LDH release compared to the RBC perfusate by 60 minutes and compared to both the RBC and control perfusates by 90 minutes. All of these findings point to less cellular damage from the PolyhHb perfusate compared to the other groups.

FIG. 12 is an exemplary graph of the wet/dry ratio for three perfusates:colloid control, RBC, and polymerized human hemoglobin. The polymerized human hemoglobin perfusate exhibited less tissue edema in comparison to the colloid control and RBC perfusates.

FIGS. 13A and 13B show (13A) the biophysical parameters of the PolyhHb synthesized at the pilot scale using a 30:1 molar ratio of glutaraldehyde to hHb compared to previous generations of exemplary commercial HBOCs and (13B) the biophysical properties for the various exemplary perfusates, wherein the hematocrit equivalent of PolyhHb was found by multiplying the PolyhHb concentration in g/dL by 3.

FIGS. 14A and 14B show lung tissue analysis of iron and Hb. FIG. 14A shows lung tissue iron as measured by the ferrozine assay, which shows the mean±S.D. for total iron quantified from tissue perfused with a control, RBCs and PolyhHb (n=6/group), FIG. 14B shows Hb immunohistochemistry of lung tissue sections after perfusion with the control perfusate at (a) 50 times magnification and (b) 630 times magnification. Lungs perfused with RBCs are shown in (c) at 50 times magnification with multiple vessels staining positive for Hb (black asterisks) and (d) at 630 times PolyHb magnification with vessel lumen Hb localized to endothelium (white asterisks). Lung tissue perfused with PolyhHb is shown in (e) at 50 times magnification and (f) at 630 times magnification showing adventitia Hb (black asterisk). Scale bars represent 500 microns (50 times magnification, images a, c, e) and 30 microns (630 times magnification, images b, d, f). Statistical analysis was performed using a One-way ANOVA with a Holm-Sidak's multiple comparisons test based on a normal distribution of data (Kolmogorov-Smirnov test). Significance was set at a p<0.05 and p values are reported as actual values to two significant figures. All analyses were performed using GraphPad Prism 9.1.1 (San Diego, California). For all images, regions of interest correspond to the same vessels in tissue sections separated by 5-micron tissue cuts. In these sections, most of the Hb from RBCs or PolyhHb would be expected to accumulate within the intravascular lumen or regions (adventitial-vasculature and macrophages) surrounding pre- and post-capillary pulmonary vessels. As expected, Hb-specific immunohistochemistry does not reveal visual reactivity within or around vessels of control perfused lung at 50 times or 630 times magnification, as shown in FIG. 14B(a) and (b). In contrast to the control perfused lungs, 14B(c) and (d) show that RBC perfused tissue demonstrates diffuse reactivity for Hb within the vascular lumen of multiple vessels (black asterisk) at 50 times magnification and at 630 times magnification near the vascular endothelium indicating a substantial build-up in cell-free Hb. Conversely, PolyhHb perfused lung tissue show visually less intra-vascular hemoglobin accumulation at 50 times magnification, but mild adventitial Hb (black asterisk) at 630 times magnification in 14B(e) and (f) respectively. This further shows the superiority of PolyhHb as the significant reduction in iron and Hb deposition compared to RBC perfused lungs demonstrates the reduced heme cytotoxicity of the next-generation PolyhHb perfusate.

FIG. 15 shows that for the PolyhHb and asanguinous perfusates, the K+ concentration of the inlet was lower than the outlet. Initially this can be explained by the Perfadex flush step that happened before NEVLP began because Perfadex has a [K⁺] of 5 mM, so residual amounts of Perfadex being removed from the organ carried a higher amount of K⁺. The opposite was true for the RBC perfusate. The RBC storage solution had a higher [K⁺] than Perfadex so the initial outlet [K⁺] was marginally lower than the inlet. The increase in [K⁺] of the RBC storage solution also explained the significantly higher K⁺ concentration compared to the other two perfusates.

FIG. 16 shows that the [Na⁺] of all exemplary species increased insignificantly over the NEVLP. Additionally, [Na+] of the PolyhHb was marginally higher than the other perfusates but this was easily explained by the fact that the PolyhHb storage buffer-modified Ringer's lactate-had an [Na⁺] of 155 mM compared to 140-145 mM for William's media

FIG. 17 shows that [Ca²⁺] remained relatively constant throughout the NEVLP. The outlet Ca²⁺ concentrations of the PolyhHb and asanguinous perfusates were lower than the inlet concentrations but this was not unexpected as increased cellular uptake of Ca²⁺ post-ischemia had been shown previously. The RBC perfusate demonstrated significantly higher [Ca²⁺] compared to the other perfusates likely due to a higher Ca²⁺ concentration in residual RBC storage buffer.

FIG. 18 shows that there was no difference between inlet and outlet Cl⁻ concentrations for any perfusate. The PolyhHb and asanguinous perfusates both remained constant throughout the NEVLP; however, the RBC perfusate demonstrated an uptick in [Cl⁻] after 60 minutes possibly correlated to the decline in lung health seen in other organ metrics at this time.

FIG. 19 shows that all three perfusates maintained a steady glucose concentration during the NEVLP. The asanguinous perfusate was the only one that demonstrated an increase in glucose concentration across the organ possibly because glucose consumption was an aerobic process and in a state of hypoxia without an O₂ carrier present, glucose uptake is significantly reduced. The PolyhHb perfusate had the lowest glucose concentration because it had the least amount of William's media in it and William's media was the sole source of glucose for these experiments.

FIG. 20 shows that both the RBC and PolyhHb perfusates showed trends for increasing lactate concentrations during the NEVLP, but only the RBC perfusate increased significantly compared to the t=0 point. This validates the results seen in the LDH results where all perfusates saw an increase in LDH, but the PolyhHb perfusate did not demonstrate a significant increase in LDH. The asanguinous control is not included in this figure because there were not enough lactate concentration data points above the lower limit of the blood gas analyzer (0.4 mM).

FIG. 21 shows that the pH of the perfusate at the outlet slightly decreased for all three perfusate between 0 and 60 minutes. After 60 minutes, the pH was stable for both the PolyhHb and the asanguinous perfusate, but the RBC perfusate began increasing in pH after this time. By the end of the NEVLP, the pH of the RBC perfusate was significantly higher than the other two perfusates. Given that lungs are supposed to be mildly acidotic, the increase in pH towards 7.4 corroborates the decline in lung function seen by other metrics at this time.

FIGS. 22A-22D shows the (22A) O₂ equilibrium curve and the (22B) O₂ offloading kinetics for PolyB1, B2, B3, B4 and hHb. Lines represent the mean from all batches. Shaded areas indicate the standard error for each PolyhHb bracket. FIG. 22C shows the pseudo-first-order Hp binding kinetics of PolyhHb/hHb. The normalized fluorescence changes (λ_{excitation}=285 nm, λₑₘᵢₛₛᵢₒₙ=310 nm) were fit to a monoexponential equation. FIG. 22D shows the second order Hp binding kinetics of PolyB1, B2, B3, B4 and hHb. The second order Hp binding rate constant was obtained via performing a linear fit of the pseudo-first-order Hp binding rate constant to the Hb concentration.

FIGS. 23A-23C show the size and MW distribution of PolyhHb and hHb. FIG. 23A shows the normalized intensity distribution of the elution time for hHb, PolyB1, B2, B3, and B4 measured using SEC-HPLC. Distributions were taken from the 413 nm absorbance normalized against the maximum intensity. FIG. 23B shows the effective diameters of hHb, PolyB1, B2, B3, and B4, measured using DLS at λ=632 nm and a 90-degree angle. FIG. 23C shows the denaturing SDS-PAGE (Novex™ (10-20%) Tris-glycine gel) of hHb, PolyB1, B2, B3, and B4, wherein DLS stands for dynamic light scattering, SDS-PAGE stands for sodium dodecyl sulfate polyacrylamide gel electrophoresis; SEC-HPLC stands for size exclusion high performance liquid chromatography, PolyhHb stands for polymerized human hemoglobin, and hHb stands for human hemoglobin.

FIG. 24 shows the biophysical properties of hHb and PolyhHb fractions, including properties related to O₂ binding, size, and Hp binding kinetics. Measured characteristics reported as average±standard deviation.

FIG. 25 shows an exemplary diagram of the cell wash and hHb purification process. RBCs were first washed over a 0.65 μm filter with cell-free hHb and cell debris permeating into the waste. After 6 diacycles of cell wash, the vessel was filled with PB to lyse the RBCs. The lysate was then passed through the 500 kDa filter to obtain purified hHb, which was then used to polymerize hHb.

FIG. 26 shows a diagram of an exemplary reactor system used for the hHb polymerization process. The reactor was filled with purified hHb on day 1, polymerized with glutaraldehyde and subsequently quenched with NaCNBH₃ on day 2 and transferred into the PolyhHb TFF purification process on day 3.

FIG. 27 shows a diagram of an exemplary 2-stage TFF PolyhHb purification process. Stage 1 retains any polymers that are too large (>0.2 μm), and stage 2 facilitates removal of unreacted chemicals and LMW Hb species from the system (<500 kDa). The PolyhHb is washed with >12 diacycles of a modified Ringer's lactate solution to remove the majority of LMW species and to buffer exchange the PolyhHb into the modified Ringer's lactate solution.

FIGS. 28A and 28B show exemplary geometry and meshing of the continuous stirred tank reactor generated by Comsol 5.3a for the PolyhHb reactor.

FIGS. 29A and 29B show the Hb concentration during the TFF-facilitated RBC washing process. Concentration of cell-free Hb in the permeate stream (29A) and concentration of cell-free Hb in the retentate vessel (29B) monitored over 6 diacycles of the RBC washing process. The cell-free Hb concentration decreased significantly over 4 diacycles in both the retentate and the permeate and remained constant from diacycle 4 to 6. Outliers are shown as black squares. A total of 5 replicates were used.

FIGS. 30A and 30B show the HCT and RBC concentration during the TFF-facilitated RBC washing process. HCT of the pooled RBC solution (30A) and cell concentration (30B) over 6 diacycles of the RBC washing process. For both the HCT and cell concentration, there was no significant difference between diacycles, indicating that there was no significant cell lysis throughout the RBC washing process. Outliers are shown as black squares. A total of 5 replicates were used.

FIG. 31 shows an exemplary OEC displaying the O₂ saturation of hHb and PolyhHb as a function of O₂ tension. The moderate P₅₀ pilot scale PolyhHb batches exhibited a significantly higher O₂ affinity compared to fully T-state pilot scale batches and bench-top scale 30:1 T-state PolyhHb batches. The OBCs are displayed as averages+/−one standard deviation shown in grey. All measurements were taken at 37° C. and pH 7.4.

FIG. 32 shows exemplary SEC-HPLC elution curves for the three types of PolyhHb discussed in this study. Bench-top scale PolyhHb batches exhibited a lower elution time, but there was a noticeable peak at 10 minutes corresponding to residual cell-free Hb, which is more completely eliminated in pilot scale batches.

FIG. 33 shows the auto-oxidation kinetics of hHb and PolyhHb. The decrease in [Fe²⁺] was linearized according to first-order rate kinetics. T-state PolyhHb—regardless of scale—exhibited a kₒₓ˜4× higher than that of the moderate P₅₀ pilot scale PolyhHb. There was no significant difference in kₒₓ between pilot and bench-top scale T-state PolyhHb.

FIG. 34 shows exemplary O₂ offloading kinetics of hHb and PolyhHb. While T-state PolyhHb trended towards a higher kₒ_{₂}_{,off}, and moderate P₅₀ PolyhHb trended towards a lower K_{O2,off}, there was no correlation found in this study. Bench-top scale T-state PolyhHb had a significantly higher k_{O}_{_{2,off}} than both pilot scale groups.

FIGS. 35A and 35B show Hp binding kinetics to hHb and PolyhHb. Representative first order kinetics for the reaction of 0.25 μM Hp with 1.25 AM PolyhHb (35A) and linearization of the apparent first order rate constant as a function of [Hb] (35B). There was no significant difference between any groups or batches of PolyhHb in either the first or second order rate kinetics. All measurements were taken at a 285 nm emission.

FIGS. 36A-36F show various turbulence parameters with streamlines in the continuous stirred tank reactor vessel. Dynamic viscosity (36A), eddy diffusivity (36B), pressure (36C), Reynold's number (36D), shear rate (36E), and vorticity (36F) in the reactor vessel were all modeled using computational fluid dynamics (CFD).

FIG. 37 shows the biophysical properties of 8 batches of pilot scale PolyhHb produced: Pilot T-state, Pilot Moderate P₅₀, Bench-top PolyhHb, and Unmodified hHb.

Reference will now be made in detail to specific aspects of the disclosed materials, compounds, compositions, formulations, articles, and methods, examples of which are illustrated in the accompanying Examples and Figures.

### DETAILED DESCRIPTION

The following description of the disclosure is provided as an enabling teaching of the disclosure in its best, currently known embodiments. Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As can be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### General Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.”

As used in the specification and the appended claims, the singular forms “a,” “an” and “the” include plural referents unless the context clearly dictates otherwise. Thus, for example, reference to “a compound”, “a composition”, or “a disorder”, includes, but is not limited to, two or more such compounds, compositions, or disorders, and the like.

It should be noted that ratios, concentrations, amounts, and other numerical data can be expressed herein in a range format. It can be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it can be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise. As used herein, the term “substantially free,” when used in the context of a composition or component of a composition that is substantially absent, is intended to refer to an amount that is then about 1% by weight or less, e.g., less than about 0.5% by weight, less than about 0.1% by weight, less than about 0.05% by weight, or less than about 0.01% by weight of the stated material, based on the total weight of the composition.

As used herein, the term “purified” or “to purify” refers to the removal of contaminants from a sample.

As used herein, the term “biological tissue sample” or “tissue sample” refers to a sample of biological tissue, including an organ.

As used herein, the term “ex vivo” refers to a medical procedure in which an organ, cells, or tissue are taken from a living body for a treatment or procedure, and then returned to the living body.

As used herein, “molecular weight” refers to the sum of the atomic masses of all atoms in a molecule, based on a scale in which the atomic masses of hydrogen, carbon, nitrogen, and oxygen are 1, 12, 14, and 16, respectively. In the case of polymeric materials, “molecular weight” can refer to weight average molecular weight.

As used herein, “ultrafiltration” refers to a variety of membrane filtration in which hydrostatic pressure forces a liquid against a membrane. Suspended solids and solutes of high molecular weight are retained, while water and low molecular weight solutes pass through the membrane. Ultrafiltration employs membranes rated for retaining solutes having a molecular weight between 1 kDa and 0.2 μm.

As used herein, “allograft” refers to the transplant of an organ, tissue, or cells from one individual to another individual of the same species.

As used herein, “hepatic artery” refers to the soft oxygenated blood vessel that supplies oxygen-rich blood to the liver, duodenum, and pancreas.

As used herein, “pulsatile flow” refers to flow with a periodic pressure fluctuation wave traveling along the flow path.

As used herein, “portal vein” refers to a blood vessel that delivers blood to the liver from the stomach, intestines, spleen, and pancreas. Most of the liver's blood supply is delivered by the portal vein.

As used herein, “continuous flow” refers to a flow in which the quantity of liquid flowing per second through any section is constant.

As used herein, “immerse” refers to submerging or placing a sample in a liquid.

As used herein, “perfuse” refers to supplying an organ or tissue, for example, with a fluid, by circulating it through blood vessels or other natural channels.

As used herein, “FiO₂” refers to fraction of inspired oxygen, which is the molar or volumetric fraction of oxygen in a gas mixture.

As used herein, “ischemic damage” refers to damage to a part of the body caused by restricted or reduced blood flow, and therefore oxygen, in a part of the body.

As used herein, “albumin” means a small globular protein with a molecular weight of 66.5 kilodaltons (kDa). It consists of 585 amino acids which are organized into three repeated homologous domains and are made up of two separate sub-domains, A and B.

As used herein, “osmolarity” refers to the concentration of a solution expressed as the total number of solute particles per liter.

As used herein, “viscosity” refers to a quantity expressing the magnitude of internal friction, as measured by the force per unit area resisting a flow in which parallel layers unit distance apart have unit speed relative to one another.

As used herein, “colloid osmotic pressure” refers to the physiochemical phenomenon that occurs when two solutions with different colloid concentrations are separated by a semipermeable membrane. It is a type of osmotic pressure induced by colloids, which can include protein and more specifically albumin, in a blood vessel's plasma that cause a pull on fluid back into the capillary.

As used herein, “glutaraldehyde” refers to C₅H₈O₂ or OCH(CH₂)₃CHO, is a transparent oily, liquid with a pungent odor. It is a dialdehyde comprised of pentane with aldehyde functions at C-1 and C-5. Alternatives to glutaraldehyde can include carboiimide, diisocyanates and polyepoxy compounds, as well as Genipin (Challenge Bioproducts Co., Ltd., Taiwan), epigallocatechin gallate (Sigma, St. Louis, MO), and grape seed proanthocyanidin (PureBulk, Inc., Roseburg, OR).

As used herein, “oxidation rate” refers to the rate at which a molecule, atom, or ion undergoes oxidation, wherein oxidation is the loss of electrons during a reaction by a molecule, atom or ion, causing the oxidation state of the molecule, atom, or ion to increase.

As used herein, “ATP” or “adenosine triphosphate” refers to the organic compound and hydrotrope that provides energy to drive many processes in living cells, including but not limited to, muscle contraction, nerve impulse propagation, condensate dissolution, and chemical synthesis. It has the chemical formula C₁₀H₁₆N₅O₁₃P₃.

As used herein, “assay” refers to the process of analyzing for the presence of a substance and the amount of that substance. In some embodiments, an assay can be performed to determine the level of ATP in a cell, which can be used to determine the viability of the cell.

As used herein, “gluconeogenesis” refers to the metabolic pathway that results in the generation of glucose from certain non-carbohydrate carbon substrates. It is present in plants, animals, fungi, bacteria, and other microorganisms. In vertebrates, gluconeogenesis can occur in the liver and to some degree in the cortex of the kidneys.

As used herein, “red blood cells” refers to the cells that carry fresh oxygen throughout the body. Red blood cells are round with a flattish, indented center, like doughnuts without a hole. Red blood cells include hemoglobin, and they are made in the bone marrow.

As used herein, “bovine hemoglobin” refers to hemoglobin from bovine blood, wherein bovine includes animals of the cattle group.

As used herein, “normothermic conditions” refers to a condition of normal body temperature. In some embodiments, normothermic conditions can include a temperature from 36° C. to 38° C.

These examples are not to be construed as limiting the sample types applicable to the present disclosure.

### Methods

The present disclosure provides for a method of preserving a biological tissue sample ex vivo. The method can include contacting the tissue sample ex vivo with a perfusion solution comprising polymerized hemoglobin, wherein the perfusion solution comprises less than 5% by weight low molecular weight hemoglobin species, based on the total weight of the perfusion solution.

As used herein, “polymerized hemoglobin”, also referred to as “polymerized Hb” or “PolyhHb”, refers to a class of hemoglobin (Hb) based O₂ carrier (HBOC) that can be synthesized and purified at large scale such that it can transport and offload O₂ to support cellular metabolism, while not demonstrating cytotoxic side-effects. In some embodiments, hemoglobin is polymerized with glutaraldehyde. As used herein, “hemoglobin species, or “hemoglobin (Hb)”, refers to the protein inside red blood cells that carries oxygen from the lungs to tissues and organs in the body and carries carbon dioxide back to the lungs. It can include four protein chains, two alpha chains and two beta chains, each with a ring-like heme group containing an iron atom. Oxygen can bind reversibly to these iron atoms and can be transported through blood.

As used herein, “perfusion solution”, also referred to as “perfusate”, refers to the solution used to perfuse a tissue sample during perfusion. In some embodiments, a perfusion solution can include PolyhHb diluted with William's cell culture media. In further embodiments, the perfusion solution can include albumin (e.g., human serum albumin). In some embodiments, the perfusion solution can include from 3 to 4 g/dL polymerized hemoglobin, 25 to 85 mM NaCl, 1 to 3 mM KCl, 6 to 20 mM KH₂PO₄, 20 to 70 mM sodium gluconate, 5 to 21 mM sodium lactate, 1 to 4 mM magnesium gluconate, 0.6 to 1.2 mM CaCl₂ dihydrate, 11 to 16 mM NaOH, 1 to 4 mM adenine, 2 to 8 mM dextrose, 0.5 to 3 mM glutathione, 2 to 8 mM HEPES, 1 to 4 mM ribose, 7 to 30 mM mannitol, 10 to 40 g/L hydroxyethyl starch, and/or 40 to 160 mg/dL N-acetyl-L-cysteine. In further embodiments, the perfusion solution can include from 3 to 4 g/dL polymerized hemoglobin, 25 to 85 mM NaCl, 1 to 3 mM KCl, 6 to 20 mM KH₂PO₄, 20 to 70 mM sodium gluconate, 5 to 21 mM sodium lactate, 1 to 4 mM magnesium gluconate, 0.6 to 1.2 mM CaCl₂) dihydrate, 11 to 16 mM NaOH, 1 to 4 mM adenine, 2 to 8 mM dextrose, 0.5 to 3 mM glutathione, 2 to 8 mM HEPES, 1 to 4 mM ribose, 7 to 30 mM mannitol, 10 to 40 g/L hydroxyethyl starch, and/or 40 to 160 mg/dl N-acetyl-L-cysteine.

As used herein, “hemoglobin species, or “hemoglobin (Hb)”, refers to the protein inside red blood cells that carries oxygen from the lungs to tissues and organs in the body and carries carbon dioxide back to the lungs. It can include four protein chains, two alpha chains and two beta chains, each with a ring-like heme group containing an iron atom. Oxygen can bind reversibly to these iron atoms and can be transported through blood.

In some embodiments, the low molecular weight hemoglobin species can have a molecular weight below 500 kDa (e.g., a molecular weight below 300 kDa). In some embodiments, the low molecular weight hemoglobin species can have a molecular weight from below 500 kDa to 400 kDa, from 400 kDa to 300 kDa, from 300 kDa to 200 kDa, from 200 kDa to 100 kDa, from 100 kDa to 50 kDa, or from 50 kDa to above 0 kDa.

In some embodiments, the perfusion solution comprises less than 5% by weight (e.g., less than 4% by weight, less than 3% by weight, less than 2% by weight, less than 1% by weight, or less than 0.5% by weight) hemoglobin species having a molecular weight below 500 kDa, based on the total weight of the perfusion solution.

In some embodiments, the perfusion solution comprises less than 5% by weight (e.g., less than 4% by weight, less than 3% by weight, less than 2% by weight, less than 1% by weight, or less than 0.5% by weight) hemoglobin species having a molecular weight below 300 kDa, based on the total weight of the perfusion solution.

In some embodiments, the polymerized hemoglobin can be prepared by a process that includes polymerizing the hemoglobin and filtering the perfusion solution by ultrafiltration against a filtration membrane having a pore size that separates the low molecular weight hemoglobin species from the polymerized hemoglobin. As used herein, “filtration membrane” refers to microporous barriers of, for example, polymeric, ceramic, or metallic materials which are used to separate dissolved materials (solutes), colloids, or find particular from solutions. A filtration membrane can be rated for retaining solutes have a specific molecular weight range from the molecular weight of one component in the solution to another component in the solution. By way of example, a filtration membrane can be rated for retaining polymerized hemoglobin with a molecular weight above that of a low molecular weight hemoglobin species (e.g., such as a membrane rated for retaining solutes have a molecular weight above 300 kDa).

In further embodiments, polymerizing hemoglobin includes adding to a Hb solution a glutaraldehyde solution over a specified period of time. In certain embodiments, polymerizing hemoglobin includes adding to a Hb solution a 0.75 wt. % glutaraldehyde solution in phosphate buffered saline (0.1 M, pH 7.4) in a 30:1 molar ratio, glutaraldehyde to Hb, over a period of 3 hours and mixing the solution for an additional hour after the glutaraldehyde addition is completed.

In some embodiments, the filtration membrane can be rated for retaining solutes having a molecular weight greater than the molecular weight of the polymerized hemoglobin, thereby forming a retentate fraction including the polymerized hemoglobin and a permeate fraction including the low molecular weight hemoglobin species. As used herein, a “retentate fraction” refers to the fraction of solution that is unable to pass through the filtration membrane. In some embodiments, the retentate fraction can include polymerized hemoglobin. As used herein, a “permeate fraction” refers to the fraction of solution that permeates the filtration membrane. In some embodiments, the permeate fraction can include low molecular weight hemoglobin species. In other embodiments, the permeate fraction can include polymerized hemoglobin and low molecular weight hemoglobin species.

In some embodiments, ultrafiltration can include tangential-flow filtration. As used herein, the term “tangential-flow filtration” refers to a process in which the fluid mixture containing the components to be separated by filtration is recirculated at high velocities tangential to the plane of the filtration membrane to reduce fouling of the filter. In such filtrations a pressure differential is applied along the length of the filtration membrane to cause the fluid and filterable solutes to flow through the membrane (e.g., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

In some embodiments, the retentate fraction including polymerized hemoglobin can have a molecular weight of greater than 300 kDa and the permeate fraction including the low molecular weight hemoglobin species can have a molecular weight of less than 300 kDa. In certain embodiments, the polymerized hemoglobin can have a molecular weight from greater than 300 kDa to 500 kDa, 300 kDa to 750 kDa, 300 kDa to 50 nm, 300 kDa to 100 nm, 300 kDa to 0.2 μm, 500 kDa to 750 kDa, 500 kDa to 50 nm, 500 kDa to 100 nm, 500 kDa to 0.2 μm, 750 kDa to 50 nm, 750 kDa to 100 nm, 750 kDa to 0.2 μm, or 50 nm to 0.2 μm. In some embodiments, the low molecular weight hemoglobin species can have a molecular weight from below 300 kDa to 200 kDa, from 200 kDa to 100 kDa, from 100 kDa to 50 kDa, or from 50 kDa to above 0 kDa.

In some embodiments, the polymerized hemoglobin can be prepared by a process that can further include filtering the retentate fraction comprising the polymerized hemoglobin by ultrafiltration against a second filtration membrane, thereby forming a second retentate fraction comprising the polymerized hemoglobin with a molecular weight above a cutoff value and a second permeate fraction comprising species having a molecular weight below the cutoff value and above 300 kDa.

In some embodiments, the cutoff value can be from 300 kDa to 0.2 μm.

In some embodiments, the cutoff value can be from 300 kDa to 500 kDa. In further embodiments, the cutoff value can be from 300 kDa to 350 kDa, 350 kDa to 400 kDa, 400 kDa to 450 kDa, or 450 kDa to 500 kDa.

In some embodiments, the cutoff value can be from 500 kDa to 750 kDa. In further embodiments, the cutoff value can be from 500 kDa to 550 kDa, 550 kDa to 600 kDa, 600 kDa to 650 kDa, 650 kDa to 700 kDa, or 700 kDa to 750 kDa.

In some embodiments, the cutoff value can be from 750 kDa to 50 nm. In further embodiments, the cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the cutoff value can from 50 nm to 0.2 μm. In some embodiments, the cutoff value can be from 50 nm to 100 nm, 100 nm to 150 nm, or 150 nm to 0.2 μm.

In some embodiments, the polymerized hemoglobin can be prepared by a process that can further include filtering the second retentate fraction including the polymerized hemoglobin by ultrafiltration against a third filtration membrane, thereby forming a third retentate fraction comprising the polymerized hemoglobin with a molecular weight above a second cutoff value and a third permeate fraction including species having a molecular weight below the second cutoff value and above the cutoff value.

In some embodiments, the second cutoff value can be from the cutoff value to 0.2 μm.

In some embodiments, the second cutoff value can be from the cutoff value to 500 kDa. In some embodiments, the second cutoff value can be from the cutoff value to 350 kDa, 350 kDa to 400 kDa, 400 kDa to 450 kDa, or 450 kDa to 500 kDa.

In some embodiments, the second cutoff value can be from 500 kDa to 750 kDa. In some embodiments, the second cutoff value can be from 500 kDa to 550 kDa, 550 kDa to 600 kDa, 600 kDa to 650 kDa, 650 kDa to 700 kDa, or 700 kDa to 750 kDa.

In some embodiments, the second cutoff value can be from 750 kDa to 50 nm. In some embodiments, the second cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the second cutoff value can be from 50 nm to 0.2 μm. In some embodiments, the second cutoff value can be from 50 nm to 100 nm, 100 nm to 150 nm, or 150 nm to 0.2 μm.

In some embodiments, the polymerized hemoglobin can be prepared by a process that can further include filtering the third retentate fraction including the polymerized hemoglobin by ultrafiltration against a fourth filtration membrane, thereby forming a fourth retentate fraction comprising the polymerized hemoglobin with a molecular weight above a third cutoff value and a fourth permeate fraction including low molecular weight hemoglobin species having a molecular weight below the third cutoff value and above the second cutoff value. In some embodiments, the third cutoff value can be from the second cutoff value to 0.2 μm

In some embodiments, the third cutoff value can be from the second cutoff value to 500 kDa. In some embodiments, the third cutoff value can be from the second cutoff value to 350 kDa, 350 kDa to 400 kDa, 400 kDa to 450 kDa, or 450 kDa to 500 kDa.

In some embodiments, the third cutoff value can be from 500 kDa to 750 kDa. In some embodiments, the third cutoff value can be from 500 kDa to 550 kDa, 550 kDa to 600 kDa, 600 kDa to 650 kDa, 650 kDa to 700 kDa, or 700 kDa to 750 kDa.

In some embodiments, the third cutoff value can be from 750 kDa to 50 nm. In some embodiments, the third cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the third cutoff value can be from 50 nm to 0.2 μm. In some embodiments, the third cutoff value can be from 50 nm to 100 nm, 100 nm to 150 nm, or 150 nm to 0.2 μm.

In some embodiments, the polymerized hemoglobin can be prepared by a process that can further include filtering the fourth retentate fraction comprising the polymerized hemoglobin by ultrafiltration against a fifth filtration membrane, thereby forming a fifth retentate fraction comprising the polymerized hemoglobin with a molecular weight above a fourth cutoff value and a fifth permeate fraction comprising species having a molecular weight below the fourth cutoff value and above the third cutoff value.

In some embodiments, the fourth cutoff value can be from the third cutoff value to 0.2 μm.

In some embodiments, the fourth cutoff value can be from the third cutoff value to 500 kDa. In some embodiments, the fourth cutoff value can be from the third cutoff value to 350 kDa, 350 kDa to 400 kDa, 400 kDa to 450 kDa, or 450 kDa to 500 kDa.

In some embodiments, the fourth cutoff value can be from 500 kDa to 750 kDa. In some embodiments, the fourth cutoff value can be from 500 kDa to 550 kDa, 550 kDa to 600 kDa, 600 kDa to 650 kDa, 650 kDa to 700 kDa, or 700 kDa to 750 kDa.

In some embodiments, the fourth cutoff value can be from 750 kDa to 50 nm. In some embodiments, the fourth cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the fourth cutoff value can be from 50 nm to 0.2 μm. In some embodiments, the fourth cutoff value can be from 50 nm to 100 nm, 100 nm to 150 nm, or 150 nm to 0.2 μm.

In some embodiments, the filtration member can be rated for retaining solutes having a molecular weight greater than 0.2 μm, thereby forming a retentate fraction including species having a molecular weight of greater than 0.2 μm and a permeate fraction including the polymerized hemoglobin having a molecular weight of less than 0.2 μm and the low molecular weight hemoglobin species.

In some embodiments, the ultrafiltration includes tangential-flow filtration. Tangential-flow filtration has a meaning as described herein.

In some embodiments, the polymerized hemoglobin can be prepared by a process that can further comprise filtering the permeate fraction including the polymerized hemoglobin and the low molecular weight hemoglobin species by ultrafiltration against a second filtration membrane, thereby forming a second retentate fraction including the polymerized hemoglobin having a molecular weight below 0.2 μm and above a cutoff value and a second permeate fraction comprising the low molecular weight hemoglobin species. In some embodiments, the polymerized hemoglobin can have a molecular weight from 50 nm to below 0.2 μm, 750 kDa to 50 nm, 500 kDa to 750 kDa, or 300 kDa to 500 kDa.

In some embodiments, the cutoff value can be from 300 kDa to 0.2 μm.

In some embodiments, the cutoff value can be from 50 nm to 0.2 μm. In some embodiments, the cutoff value can be from 50 nm to 100 nm, 100 nm to 150 nm, or 150 nm to 0.2 μm.

In some embodiments, the cutoff value is from 750 kDa to 50 nm. In some embodiments, the cutoff value can be from 750 kDa to 800 kDa, 800 kDa to 850 kDa, 850 kDa to 900 kDa, 900 kDa to 950 kDa, 950 kDa to 1000 kDa, 1000 kDa to 25 nm, or 25 nm to 50 nm.

In some embodiments, the cutoff value is from 500 kDa to 750 kDa. In some embodiments, the cutoff value can be from 500 kDa to 550 kDa, 550 kDa to 600 kDa, 600 kDa to 650 kDa, 650 kDa to 700 kDa, or 700 kDa to 750 kDa.

In some embodiments, the cutoff value is from 300 kDa to 500 kDa. In some embodiments, the cutoff value can be from 300 kDa to 350 kDa, 350 kDa to 400 kDa, 400 kDa to 450 kDa, or 450 kDa to 500 kDa.

In some embodiments, the tissue sample can include an organ. As used herein, “organ” refers to a collection of tissues that structurally form a functional unit specialized to perform a particular function. Organs can be defined by a shape, location in the body, and or a function. In some embodiments, organs can include, but are not limited to, a kidney, liver, or heart.

In some embodiments, the tissue sample can include one or more of a heart, lung, liver, kidney, pancreas, small intestine, limb, or portion thereof.

In some embodiments, the tissue sample can include an allograft for transplantation.

In some embodiments, contacting the tissue sample ex vivo with a perfusion solution can include immersing the tissue sample partially or completely within the perfusion solution.

In some embodiments, contacting the tissue sample ex vivo with a perfusion solution can include perfusing the tissue sample with the perfusion solution.

In some embodiments, contacting the tissue sample ex vivo with a perfusion solution can include delivering at least a portion of the perfusion solution to the tissue sample in a pulsatile flow.

In some embodiments, the perfusion solution can be oxygenated with a gas mixture having an FiO₂ concentration from 1% to 100% by weight of O₂ in the gas mixture. In further embodiments, the FiO₂ concentration can be from 1% to 10%, 10% to 21%, 21% to 40%, or 40% to 80% by weight of O₂ in the gas mixture.

In some embodiments, the method can further comprise perfusing the organ prior to transplantation, perfusing the organ during surgery or treatment, or perfusing the organ prior to or during collection of cells from the organ.

In some embodiments, the method can further comprise maintaining the tissue at a temperature of from 18° C. to 37° C. In further embodiments, the method can further comprise maintaining the tissue at a temperature of from 18° C. to 22° C., 22° C. to 25° C., 25° C. to 28° C., 28° C. to 30° C., 30° C. to 33° C., 33° C. to 35° C., or 35° C. to 37° C.

In some embodiments, the method can further comprise maintaining the tissue at a temperature of from 35° C. to 37° C.

In some embodiments, the tissue sample can have ischemic damage.

In some embodiments, the perfusion solution can comprise from 1% by weight to 5% by weight albumin, based on total weight of the perfusion solution.

In some embodiments, the perfusion solution can have an osmolarity from 270 to 370 mOsm. In further embodiments, the perfusion solution can have an osmolarity from 270 to 290 mOsm, 290 to 310 mOsm, 310 to 330 mOsm, 330 to 350 mOsm, or 350 to 370 mOsm.

In some embodiments, the perfusion solution can have a viscosity from 2 cP to 4.5 cP at normothermic conditions. In further embodiments, the perfusion solution can have a viscosity from 2 cP to 3 cP, 3 cP to 4 cP, or 4 cP to 4.5 cP.

In some embodiments, the perfusion solution can have a viscosity from 2.9 to 3.7 cP at normothermic conditions. In further embodiments, the perfusion solution can have a viscosity from 2.9 to 3.1 cP, 3.1 to 3.3 cP, 3.3 to 3.5 cP, or 3.5 to 3.7 cP.

In some embodiments, the perfusion solution can have a colloid osmotic pressure from 14 mm Hg to 20 mm Hg. In further embodiments, the perfusion solution can have a colloid osmotic pressure from 14 mm Hg to 16 mm Hg, 16 mm Hg to 18 mm Hg, or 18 mm Hg to 20 mm Hg.

In some embodiments, the perfusion solution can have a colloid osmotic pressure from 16.8 mm Hg to 17.6 mm Hg.

In some embodiments, the polymerized hemoglobin can be synthesized using a molar ratio from 20:1 to 40:1 of glutaraldehyde to hemoglobin. In some embodiments, the polymerized hemoglobin can be synthesized using a molar ratio from 25:1 to 35:1 of glutaraldehyde to hemoglobin. In further embodiments, the ratio can be at least 1:1 (e.g., at least 2:1, at least 5:1, at least 10:1, at least 20:1, at least 30:1, at least 40:1, at least 50:1, at least 60:1, at least 70:1, at least 80:1, or at least 90:1). In certain embodiments, the ratio can be 100:1 or less (e.g., 90:1 or less, 80:1 or less, 70:1 or less, 60:1 or less, 50:1 or less, 40:1 or less, 30:1 or less, 20:1 or less, or 10:1 or less).

The range can vary between any of the minimum values described above to any of the maximum values described above. For example, the polymerized hemoglobin can be synthesized at a molar ratio that can range from 1:1 to 100:1 (e.g., from 1:1 to 10:1, 10:1 to 20:1, 20:1 to 30:1, 30:1 to 40:1, 40:1 to 50:1, 50:1 to 60:1, 60:1 to 70:1, 70:1 to 80:1, 80:1 to 90:1, 90:1 to 100:1).

In some embodiments, the partial pressure of oxygen at which 50% of the polymerized hemoglobin is saturated with oxygen can be from 1 mm Hg to 50 mm Hg. In further embodiments, the partial pressure of oxygen at which 50% of the polymerized hemoglobin is saturated with oxygen can be from 10 mm Hg to 12 mm Hg, 12 mm Hg to 14 mm Hg, 14 mm Hg to 16 mm Hg, 16 mm Hg to 18 mm Hg, or 18 mm Hg to 20 mm Hg. In some embodiments, the partial pressure of oxygen at which 50% of the polymerized hemoglobin is saturated with oxygen can be from 14 mm Hg to 16 mm Hg. As used herein, saturation with oxygen refers to the binding of heme in hemoglobin with oxygen molecules. The degree of oxygen saturation of hemoglobin is dependent on the number of heme units that are bound to oxygen (e.g., 50%) saturation with oxygen means that half of the heme units are bound to oxygen molecules).

In some embodiments, the polymerized hemoglobin can exhibit an oxidation rate of from 0.0020 to 0.0085 h⁻¹. In further embodiments, the polymerized hemoglobin can exhibit an oxidation rate of from 0.0020 to 0.0045 h⁻¹, 0.0045 to 0.0065 h⁻¹, or 0.0065 to 0.0085 h⁻¹. In some embodiments, the polymerized hemoglobin can exhibit an oxidation rate of from 0.0045 to 0.0065 h⁻¹.

The present disclosure also provides a method of assessing viability of a biological tissue sample that can include measuring at least one energy parameter and determining a measure of viability as a function of the at least one energy parameter. As used herein, “viability” refers to the suitability of biological tissue samples for transplantation. If a tissue sample is not considered viable for transplantation, it cannot be used for organ transplantation. As used herein, an “energy parameter” refers to a characteristic of a tissue sample that indicates the energy content of the tissue sample. In some embodiments, an energy parameter can include the level of energy substrates of the tissue sample, such as ATP.

In some embodiments, determining a measure of viability can include comparing a measured energy parameter to a threshold representative of a transplantability threshold of the tissue sample. As used herein, “transplantability threshold” refers to a parameter or characteristic that must be met or exceeded in order for a tissue sample to be suitable for transplantation into a subject. In some embodiments, a transplantability threshold can include a required level of energy substrate in the tissue sample, a level of cellular energy status, a level of a plurality of metabolites, a level of oxygen consumption by the tissue sample, a level of gluconeogenesis, or a measurement level of nitrogen metabolism by the tissue sample.

In some embodiments, measuring at least one energy parameter can include assaying for the level of ATP, measuring the cellular energy status during normothermic perfusion of the tissue sample, measuring the level of a plurality of metabolites, measuring the oxygen consumption by the tissue sample, measuring the level of gluconeogenesis of the tissue sample, or measuring the nitrogen metabolism by the tissue sample. As used herein, “cellular energy status” refers to the energy state of the cells in the tissue sample as regulated by ATP. It can be an indicator of cell health and can also contribute to the regulation of signaling activities.

In some embodiments, the method can further comprise storing the tissue sample prior to assessing its viability. In some embodiments, storing the tissue sample can include cooling and storing at a predetermined sub-zero temperature without freezing. As used herein, “storing” is used to refer to the process of preserving a tissue sample between harvesting it from the providing subject and transplanting it into the receiving subject. In some embodiments, storing can include cold storage, such as storage in a sterile container on wet ice.

In some embodiments, the tissue sample can be contacted with a media including a supercoolant agent prior to cooling and freezing. As used herein, “media” can include supercoolant agents, low-potassium medium, antioxidants, bioregulators, such as CO, H₂S, or NO, or any combination thereof. As used herein, “supercoolant agents” are substances used when preserving tissue sample that aid in avoiding antifreeze toxicity. In some embodiments, a supercoolant agent can include 3-O-methyl-glucose (30 MG).

Also provided are methods of enhancing the performance of a composition comprising red blood cells in tissue storage or perfusion. These methods can comprise comprising adding a perfusion solution described herein to the oxygen carrier comprising the red blood cells. The polymerized hemoglobin present in the perfusion solution can improve the ability of the composition comprising red blood cells to oxygenate the tissue.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures and other reaction ranges and conditions that can be used to optimize the product purity and yield obtained from the described process. Only reasonable and routine experimentation will be required to optimize such process conditions.

### Example 1. Polymerized Human Hemoglobin Based Oxygen Carrier for Maintaining Lung Viability During Normothermic Ex Vivo Lung Perfusion (NEVLP)

### Overview

Normothermic ex vivo lung perfusion (NEVLP) is able to resuscitate marginal lung allografts to make more organs available for transplantation. During normothermic perfusion, cellular metabolism is active, creating a need for an oxygen (O₂) carrier to adequately oxygenate the graft. As an O₂ carrier, red blood cells (RBCs) are prone to hemolysis in perfusion circuits leading to cell-free hemoglobin (Hb) toxicity, oxidative tissue damage, and cell death. Polymerized human Hb (PolyhHb) is a class of Hb-based O₂ carrier (HBOC) that can be synthesized and purified at a large scale such that it can transport and offload O₂ to support cellular metabolism, while not demonstrating cytotoxic side-effects. In this example, a next-generation PolyhHb was synthesized and purified at the 30 L pilot scale with a suite of analytical techniques including size exclusion chromatography high pressure liquid chromatography (SEC-HPLC) to validate that the majority of low molecular weight Hb polymers (<500 kDa) were removed from the solution. The PolyhHb was then added to an existing colloid solution and compared to both RBC and asanguinous perfusates in a rat NEVLP model. The pulmonary artery pressure and pulmonary vascular resistance were both higher in lungs perfused with RBCs, likely due to vasoconstriction from hemolysis and subsequent exposure to cell-free Hb. Lungs perfused with PolyhHb also demonstrated greater oxygenation than those perfused with RBCs and elicited less cellular damage and edema than both other perfusates.

### Background

Normothermic machine perfusion (NMP) has shown promise in the field of organ storage and preservation to improve the viability of transplanted organs. Studies have shown that NMP can resuscitate ECD organs to obtain pre-transplant quality comparable to nonmarginal organs for subsequent transplantation. There is a significant metabolic demand maintaining organs at normothermia and this metabolic demand is only exacerbated in DCD organs. There is a demand for an artificial RBC substitute that can store and transport oxygen (O₂) to meet the metabolic demands of perfused organs while not being subject to hemolysis or Hb/home/iron toxicity.

Of all the different types of HBOCs being studied to carry and offload O₂ to surrounding tissue, polymerized Hb (PolyHb) has the ability to be synthesized and purified at large scale. Any adverse side-effects are the result of the presence of cell-free Hb and low molecular weight (MW) Hb polymers (<500 kDa) that extravasate out of the circulation into the tissue space, which leads to nitric oxide (NO) scavenging and subsequent vasoconstriction, systemic hypertension, and oxidative tissue injury. Elimination of these low MW Hb species from the NMP perfusate may mitigate these deleterious side-effects. In ex vivo lung perfusion (EVLP), there is heightened concern about the development of edema which further necessitates the need for development of a perfusate that will not damage the especially delicate tissues of the lung. Currently, the majority of EVLPs, such as those that follow the Toronto Protocol, contain no O₂ carrying molecule in the perfusate. Without an O₂ carrier in the perfusate, the metabolic demand during normothermic EVLP (NEVLP), especially when perfusing DCD lungs, is not fully met.

This example utilizes a clinically relevant, validated, lung DCD model and NEVLP platform to assess the ability of a polymerized human Hb (hHb) (PolyhHb) HBOC perfusate to meet DCD organ metabolic demands and evaluate organ quality as compared to an RBC and asanguinous perfusate.

### Materials and Methods

Materials. Glutaraldehyde (70%), sodium chloride (NaCl), potassium chloride (KCl), sodium hydroxide (NaOH), sodium dithionite (Na₂S₂O₄), calcium chloride (CaCl₂)₂H₂O), sodium lactate, Nacetyl-L-cysteine (NALC), sodium cyanoborohydride (NaCNBH₃), sodium phosphate dibasic (Na₂HPO₄), sodium phosphate monobasic (NaH₂PO₄), trichloroacetic acid (C₂HCl₃O₂), sodium acetate (C₂H₃NaO₂), ascorbic acid (C₆H₈O₆), and citrate buffer were purchased from Sigma-Aldrich (St. Louis, MO). Hollow fiber tangential flow filtration (TFF) modules (polyethersulfone (PES) 0.2 μm and polysulfone (PS) 500 kDa) were purchased from Spectrum Laboratories (Rancho Dominguez, CA). All other chemicals were purchased from Fisher Scientific (Pittsburgh, PA). Expired human RBC units were generously donated by Canadian Blood Services, Ottawa, Canada.

William's E Medium (A12176-01) was purchased from Gibco, Thermo Fisher Scientific (Waltham, MA). Sprague-Dawley rat RBCs were purchased from Innovative Research Inc. (Novi, MI). Male Sprague-Dawley rats were purchased from Envigo (Indianapolis, IN) and housed under pathogen-free conditions at The Ohio State University Animal Facility. All procedures were humanely performed according to the NIH and the National Research Council's Guide for the Humane Care and Use of Laboratory Animals and with the approval of The Ohio State University Institutional Animal Care and Use Committee (IACUC Protocol #2012A00000135-R2)

PolyhHb Synthesis. The PolyhHb was produced from a single pilot batch. Twenty units of expired human RBCs were added to 7 L of 0.9 wt % saline to achieve a pooled hematocrit of 22%. RBCs were washed with 6 volume difiltrations of saline over a 0.65 μm modified polyethersulfone (mPES) TFF filter. The RBC solution was then concentrated down to 10 L before being lysed for 1 hour with an equal volume of phosphate buffer (PB) (3.75 mM, pH 7.4). Cell debris was eliminated by filtration of the RBC lysate through a 500 kDa PES filter with purified Hb and other intracellular proteins present in the permeate flowing into a 30 L bioreactor. 470 g of Hb was allowed to flow into the bioreactor before the permeate feed line was closed. The Hb in the bioreactor had deionized water, NaCl, and KCl added to it to bring the final solution to 18.2 g_{Hb}/L in phosphate buffered saline (PBS). The bioreactor was held under a nitrogen (N₂) head at 14° C. overnight.

The next morning, the cooling coils were turned off and the bioreactor was brought to and maintained at 37° C. using a heating jacket. The Hb solution was deoxygenated using an external flow loop with a G420 X40 gas-liquid exchanger (3M, Maplewood, MN) and N₂ as the sweep gas. Once the partial pressure of O₂ (pO₂) reached below 5 mm Hg on a Rapidlab 248 (Siemens, Malvern, PA) blood gas analyzer (BGA), a bolus of 6 g of Na₂S204 was added to reduce the pO₂ to below readable levels on the BGA. Upon full deoxygenation of the solution, a 0.75 wt % glutaraldehyde solution in PBS (0.1 M, pH 7.4) was added in a 30:1 molar ratio glutaraldehyde to hemoglobin over a period of 3 hours. The solution was allowed to mix for an additional hour after glutaraldehyde addition was completed. After polymerization was completed, the heating jacket was removed and the cooling coils were turned back on. NaCNBH₃ was used to quench the reaction at a 7:1 molar ratio of NaCNBH₃ to glutaraldehyde through rapid delivery of a 3.5 wt % solution of NaCNBH₃ in PBS (0.1 M, pH 7.4). The solution continued to quench and cool overnight.

PolyhHb purification followed methods described previously in the literature. The sole deviation was that the number of diafiltrations was increased from 8 to 16 to ensure the final PolyhHb product contained <5% low MW Hb species (<500 kDa). The final product was concentrated to 11.5 g/dL and stored at −80° C.

Protein Quantification. The total Hb concentration and percentage of methemoglobin (metHb) were determined using the cyanomethemoglobin method. This assay was used to quantify PolyhHb during production, after purification, and during NEVLP. Cell-free Hb in RBC perfusates was quantified by centrifuging the perfusate until the RBCs formed a pellet. The resulting supernatant was then analyzed via the cyanomethemoglobin method. To determine the total Hb concentration in RBCs, RBCs were lysed through freeze thaw cycles followed by dilution in PB (3.75 mM, pH 7.4). The lysed RBCs were again centrifuged down to a pellet and the supernatant assayed for the total Hb concentration.

Biophysical Parameters. The oxygen equilibrium curve, MW distribution, and auto-oxidation kinetics for PolyhHb and Hb were all measured as previously described in the literature. Briefly, oxygen equilibrium curves were measured using a Hemox Analyzer (TCS Scientific, New Hope, PA) at 37° C. and pH 7.4. The MW distribution was estimated by performing size exclusion high pressure liquid chromatography (SEC-HPLC) using an Acclaim SEC-1000 column (Thermo Scientific, Waltham, MA) on a Thermo Scientific UHPLC System using MW standards. Auto-oxidation kinetics were measured via UV-visible spectrometry over 24 hours at 37° C. The solution viscosity was measured using a DV3T-CP cone and plate viscometer (Brookfield AMETEK, Middleboro, MA), and osmolarity was measured using a Gonotech 010 freezing point osmometer (Gonotech GmbH, Berlin, Germany). Colloid osmotic pressure (COP) was measured using a Wescor 4420 Colloid Osmometer (Wescor, Logan, UT).

Perfusate Formulation. Perfusates were formulated using William's cell culture media as the primary fluid at a final volume of 165 mL. For the HBOC perfusate, PolyhHb was diluted with William's media to a final concentration of 3.7±0.1 g/dL. Twenty-five percent human serum albumin (HSA) was added such that the final perfusate consisted of 3% HSA by weight. Rat RBCs were diluted to a final hematocrit of 15% to be in line with the Lund protocol. The asanguinous control consisted of 4% HSA in William's media. Each of these perfusates was brought to 37° C. and pH 7.4 using THAM buffer before beginning NEVLP.

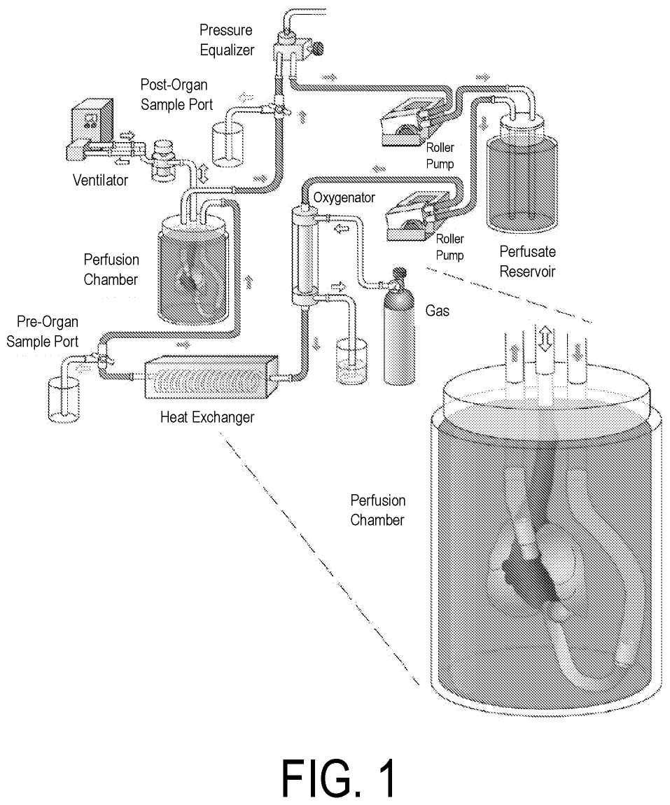

NEVLP. Briefly, rats were fully anesthetized by a ketamine and xylazine injection via intraperitoneal injection then shaved and positioned for lung procurement. The incision was started by cutting the abdomen to open the peritoneum. Heparin (100 IU/kg) was injected via the inferior vena cava (IVC) and allowed to circulate for 10 minutes. Rats were then connected to the ventilator via tracheostomy and exsanguinated by cutting the IVC. The thoracic cavity was opened, and the pulmonary vein and left atrium were cannulated via transapical approach. The heart-lung bloc was removed from the chest cavity with the lung-no-touch technique before being connected to the ex vivo perfusion system as shown in FIG. 1.

Lungs were ventilated for 2 hours or until pulmonary artery pressure (PA pressure) exceeded 100 cm H₂O. Ventilation was performed with ambient air at 60 bpm and tidal volume of 4 mL/kg of rat with a positive end expiratory pressure (PEEP) of 2 cmH₂O. The perfusion flow rate was set at 20% of estimated cardiac output (75 mL/kg of rat). Perfusate samples were collected at each timepoint of NEVLP and snap frozen until needed for analysis.

Post-NEVLP Analysis. Lactate dehydrogenase (LDH) released into the perfusate was measured using a LDH cytotoxicity detection kit (Clontech Laboratories, Mountain View, CA) and following the manufacturer's instructions. The right inferior lobe was used for wet to dry ratio determination. The lobe was weighed immediately upon perfusion termination for wet weight, dried at 60° C. for 48 hr., and then weighed again for the dry weight.

Lung Histology. Snap frozen superior lung lobe samples were homogenized in 10 volume equivalents of deionized water. 1 mL of the homogentate was incubated (60 min, 50° C.) with 500 μL of a solution containing 1 M hydrochloric acid (HCl) and 10% trichloratic acid. Samples were then centrifuged at 8,000 g for 15 minutes and 375 μL of supernatant was mixed with 125 μL of a 2% ascorbic acid solution to reduce ferric iron. To quantify, ferrous iron 100 μL of a solution containing 1 g/L of FerroZine™ (ACROS Orgnaics, Geel, Belgium) and 1.5 M sodium acetate was added to the aliquot. Samples were allowed to develop for 30 minutes, after which the absorbance at 562 nm was measured using a BioTek Synergy HTX plate reader.

Paraffin embedded tissue blocks from the lung middle lobe were prepared, and tissue was sectioned (5-micron thickness) by the University of Maryland Baltimore Histology Core. Sections were dewaxed and hydrated in graded ethanol percentages. Heat-mediated antigen retrieval was performed using pH 6.0 citrate buffer. The solution was brought to a boil, and then allowed to cool for 30 minutes. Once cool, sections were incubated at room temperature for 30 minutes with 3% horse serum. Sections were then incubated overnight at 4° C. with an antibody against the Hb α-chain (1:300, Abcam, Ab92492). A biotinylated secondary antibody was used for Hb α-chain staining with sections incubated for 30 minutes (1:300, Invitrogen, 31820). Sections were then incubated for 30 minutes with avidin and biotinylated horseradish peroxidase (VECTASTAIN Elite® ABC Kit, Vector Laboratories), and incubated for 3 minutes with 3,3′-diaminobenzidine and H₂O₂ (SIGMAFAST™, Sigma). Sections were then counterstained in hematoxylin (Gil no. 2, Fisher). All images were obtained using a Leica DM4-B TL bright field microscope (Leica, Wetzlar, Germany) and captured with LAS X software at 630 times total magnification using a 63 times objective withoil immersion at 22.02 milliseconds of exposure.

Statistical Analysis. All statistical analysis was done using RStudio (Version 1.4.1106, RStudio, Inc., Boston, MA) using a one-way ANOVA to determine significance. Significance was reported to an α value of 0.05. In all figures, an asterisk is used to indicate significance between groups. A pound sign is used to indicate significance within a group compared to its initial value.

### Results and Discussion

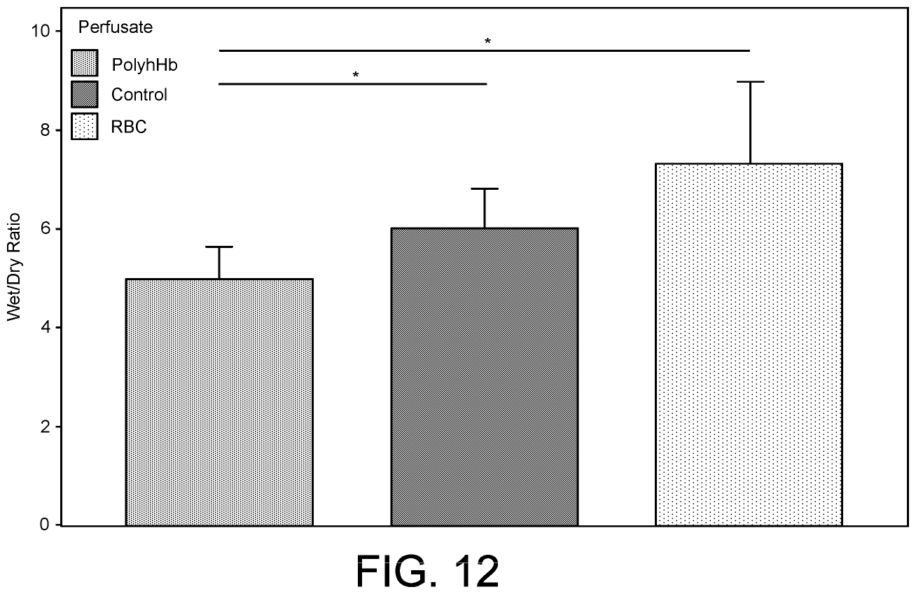

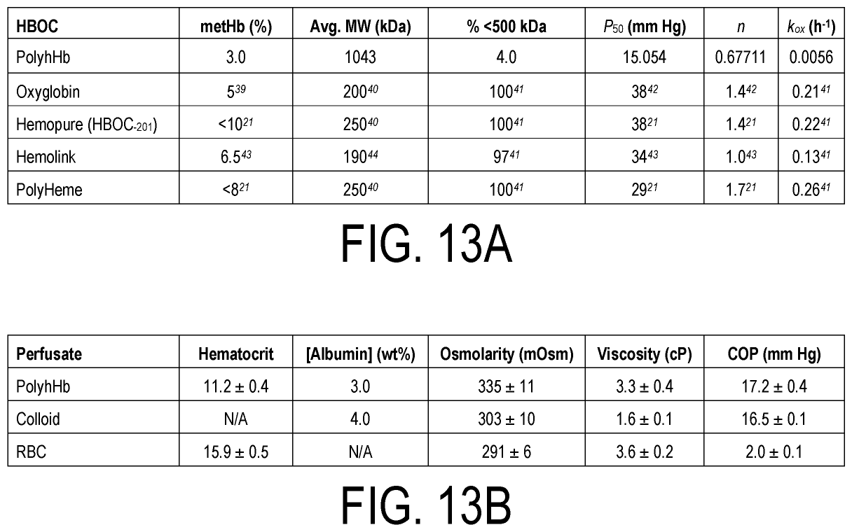

PolyhHb Biophysical Properties. The potential ability of the PolyhHb synthesized in this study to function as a perfusate can be assessed by a variety of in vitro parameters. The metHb level (%), average MW, pO₂ at which 50% of the Hb or PolyhHb is saturated with 02 (P₅₀), cooperativity coefficient (n), percentage of low MW species in solution (<500 kDa), and auto-oxidation rate constant (kₒₓ) are listed in FIG. 13A for both the PolyhHb produced in this example as well as for prior generations of commercial HBOCs.

The percentage of metHb in the PolyhHb produced in this study is marginally lower than other high-MW PolyhHbs. This can likely be explained by the significantly lower kₒₓ compared to previous generations of commercial HBOCs. Hemolink (Hemosol Inc.), Hemopure (Biopure Corp.), Oxyglobin (Biopure Corp.), and PolyHeme (Northfield Laboratories) are all commercial polymerized Hbs that failed Phase III clinical trials and each of these HBOCs were reported to have a kₒₓ between 0.13-0.26 h⁻¹ which represents an auto-oxidation rate 20-40 times faster than the PolyhHb produced in this example. The lower rate of auto-oxidation means that in an NEVLP circuit, the HBOC formulation will retain its ability to load and offload oxygen as intended over a longer period of time, since most of the Hb will exist in the ferrous form (HbFe²⁺) instead of the oxidized ferric form (HbFe³⁺, metHb) which cannot bind oxygen. This is invaluable in NEVLP experiments because prior HBOCs can lose a third of their O₂-carrying capacity in as little as an hour during NMP.

The average MW of the pilot scale PolyhHb preparation is 5 times larger than prior generations of commercial HBOCs. Additionally, the PolyhHb produced in this example only contains a fraction of the low MW species (<500 kDa) compared to previous generation of commercial HBOCs. As mentioned above, the high concentrations of Hb and low MW Hb polymers in prior generations of commercial HBOCs have created a host of disqualifying problems preventing them from being successful in transfusion medicine despite their ability to carry and offload O₂ as intended.

The P₅₀ and n are lower for this PolyhHb compared to previous generations of commercial HBOCs, which were synthesized in the low O₂-affinity tense quaternary state (T-state). These prior HBOCs were d readily offloaded O₂ to surrounding tissue to support metabolic activity. The indiscriminate offloading of O₂ can actually be detrimental to the organ due to a phenomenon called autoregulation, whereby in the presence of high O₂ levels, the organism limits oxygen consumption in an attempt to keep tissue oxygen levels constant. Additionally, too much oxygen offloading during perfusion has been shown to generate reactive oxygen species (ROS) which are highly detrimental to graft survivability. Therefore, it is more beneficial to design an HBOC with an oxygen affinity similar to or less than that of RBCs in order to support metabolic activity without inducing autoregulation or ROS formation. Hence, the P₅₀ of the PolyhHb described in this study being half that of previous generations of commercial HBOCs may actually prove to be beneficial given that the P₅₀ of human RBCs is about 26 mmHg.

NEVLP. In order for the full potential of NEVLP to be realized, an oxygen carrier needs to be present in the perfusate in order to support the metabolic activity of the lung during NMP. The PolyhHb perfusate concentration remains very stable throughout the perfusion. There is an insignificant increase in concentration due to evaporative concentration of the PolyhHb in the NEVLP circuit; however, no point is significantly different compared to the initiation of NEVLP.

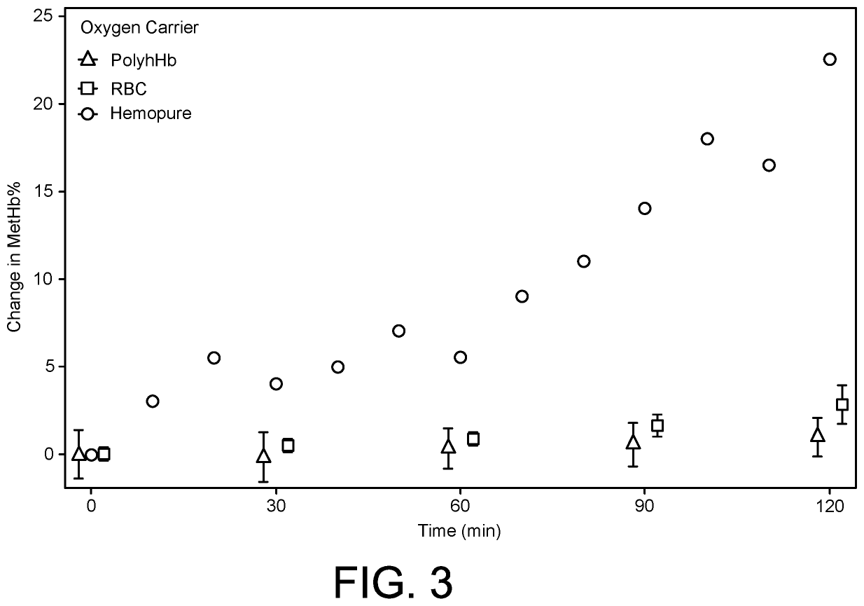

As shown in FIG. 13A, the PolyhHb synthesized in this work exhibited exceptionally lower kₒₓ compared to any previously synthesized polymerized Hb. This holds true beyond in vitro experiments as demonstrated in FIG. 3. The PolyhHb perfusate demonstrated stability throughout the course of NEVLP, increasing the percentage of metHb by less than 2%. Compared to previously published HBOC perfusate data, shown in FIG. 3, which demonstrated an almost 25% increase in metHb in the perfusate. In this previous study, the group administered multiple bolus injections of vitamin C and glutathione in an effort to reduce the metHb being formed, but even with these interventions, almost a third of the final HBOC-containing perfusate was in the non-O₂ carrying ferric state (i.e. metHb) after just two hours of NMP. The rapid oxidation of Hb is ameliorated by the PolyhHb synthesized in this study, which facilitates the possibility of extended length perfusions that have not previously been viable while also reducing the rate of ROS formation by minimizing the fraction of Hb in the ferric state.

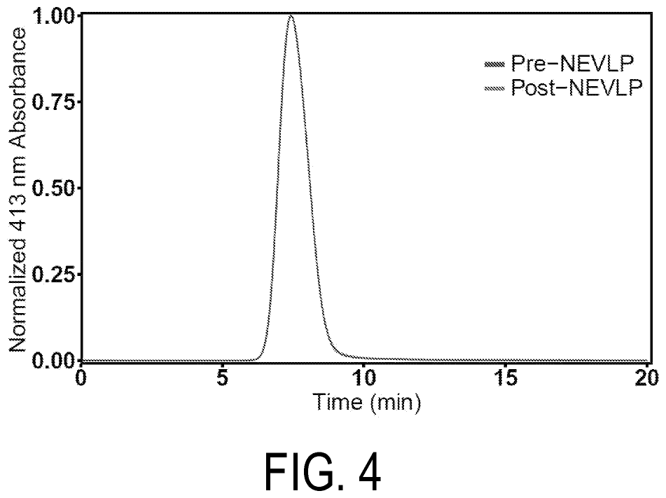

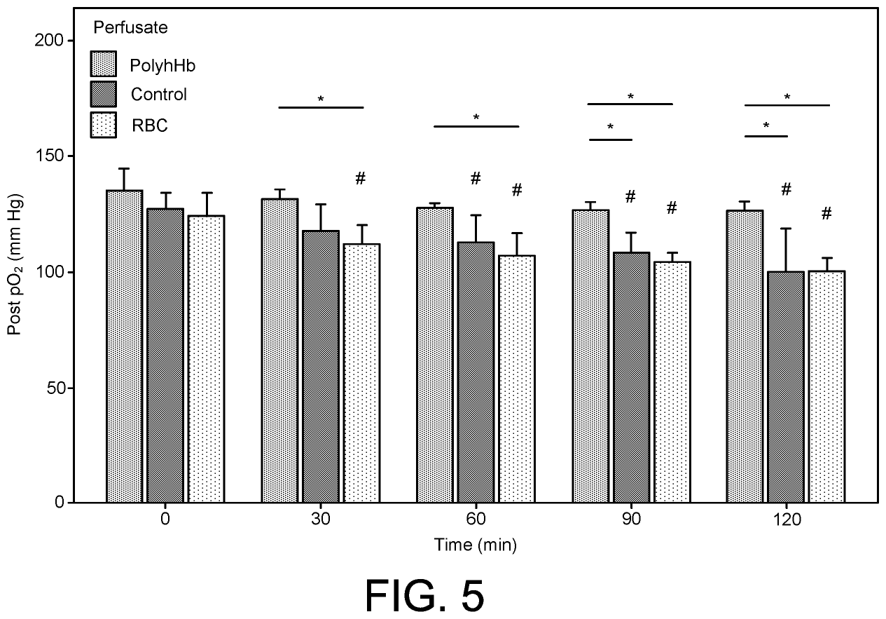

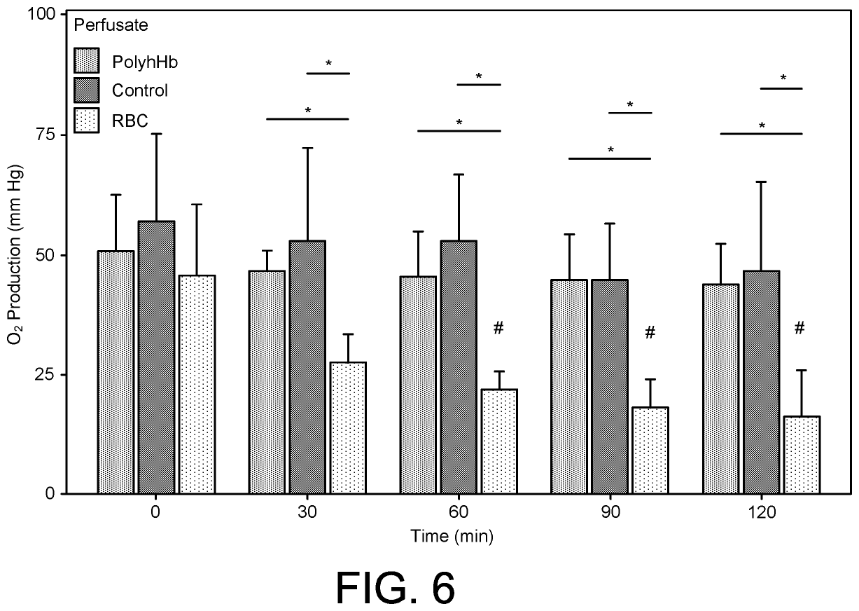

Another shortcoming of previous generations of commercial HBOCs is their propensity for low molecular weight Hb species (<500 kDa) extravasation out of circulation into the tissue space, which has detrimental side-effects that were previously elaborated on. To validate that the PolyhHb is not extravasating out of circulation, SEC-HPLC was run on the PolyhHb perfusate both before and after NEVLPs. These results are shown in FIG. 4 and the pre- and post-NEVLP curves overlap perfectly. This proves that PolyhHb does not extravasate into the tissue space during NEVLP thus increasing the safety and efficacy of this next-generation PolyhHb synthesized in this current example compared to prior commercial HBOCs.

The organ metrics measured during NEVLP demonstrated equal or superior graft performance in lungs perfused with PolyhHb compared to both the asanguinous control as well as the RBC perfusate. The pO₂ of the post-bloc perfusate demonstrates the graft's ability to supply O₂ to the system and eventually potentially to circulating blood in a transplant recipient. As such, maintaining an adequate post-bloc pO₂ is an important indicator of graft health. FIG. indicates that the PolyhHb perfusate best preserves the amount of O₂ that the lung is able to deliver to the system. After 30 minutes of NEVLP, lungs perfused with PolyhHb were delivering significantly more O₂ than lungs perfused with RBCs. At 90 minutes of NEVLP, PolyhHb perfused lungs were also delivering more O₂ than those perfused with the colloid control. Additionally, by 60 minutes of NEVLP, lungs perfused with both RBCs and the control experienced a significant decrease in oxygenation compared to their initial values. The lungs perfused with PolyhHb did not experience a statistically significant decrease in their ability to deliver Oz. By this metric, PolyhHb was able to sustain graft health better than both other perfusates,

The O₂-carrying ability of PolyhHb was likely the driving factor for this behavior. The increase in post-bloc pO₂ for the asanguinous control compared to PolyhHb can partially be explained by the increase in oxygenation potential of the perfusate. Having an O₂ carrier in solution will inherently facilitate more O₂ to be stored and transported in the perfusate compared to only using a colloid. Given that grafts perfused with RBCs also had an O₂ carrier in the perfusate, the improvement in PolyhHb lungs over RBC lungs can be attributed to an increase in graft health. Not only did lungs perfused with PolyhHb have a higher post-bloc pO₂, but they also did not experience a significant decrease in pO₂ over time. This also can only be attributed to an increase in graft health. Taken together, the PolyhHb perfusate sustained the ability of the lungs to provide O₂ to the perfusate better than either RBCs or an asanguinous solution.

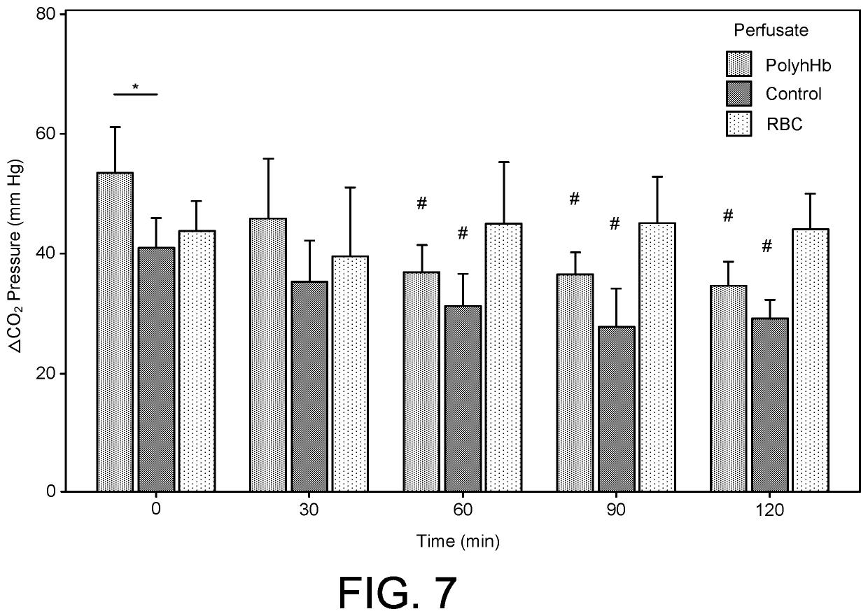

In addition to facilitating O₂ delivery to the perfusate, the other primary function of the lungs is carbon dioxide (CO₂) clearance. FIG. 7 shows the change in CO₂ tension across the heart-lung bloc as a function of time. There was no significant difference between groups after the initial time point; however, both the PolyhHb and colloid control perfusates exhibited less CO₂ clearance after 60 minutes. The RBC perfusate did not experience this decline. The colloid control was therefore the only perfusate that saw a reduction in gas exchange properties of the lungs with respect to both O₂ and CO₂.

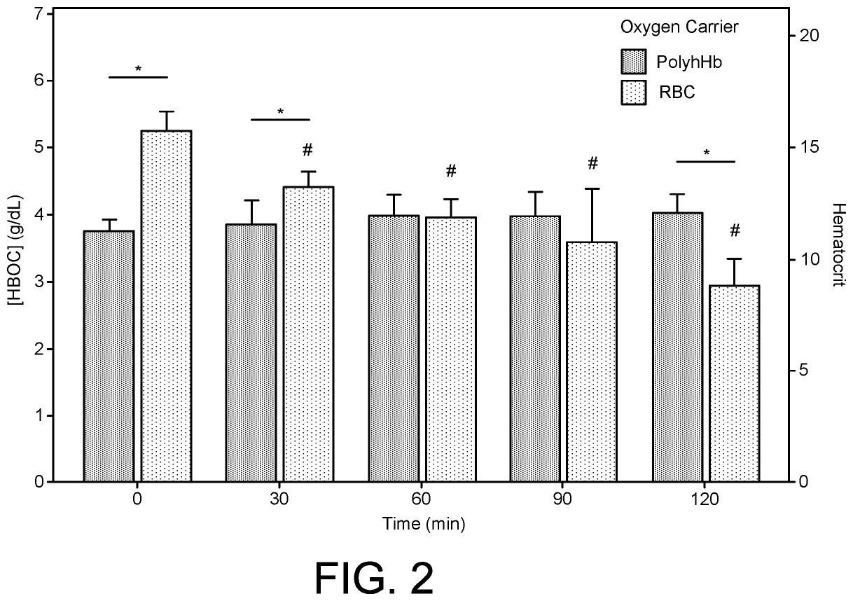

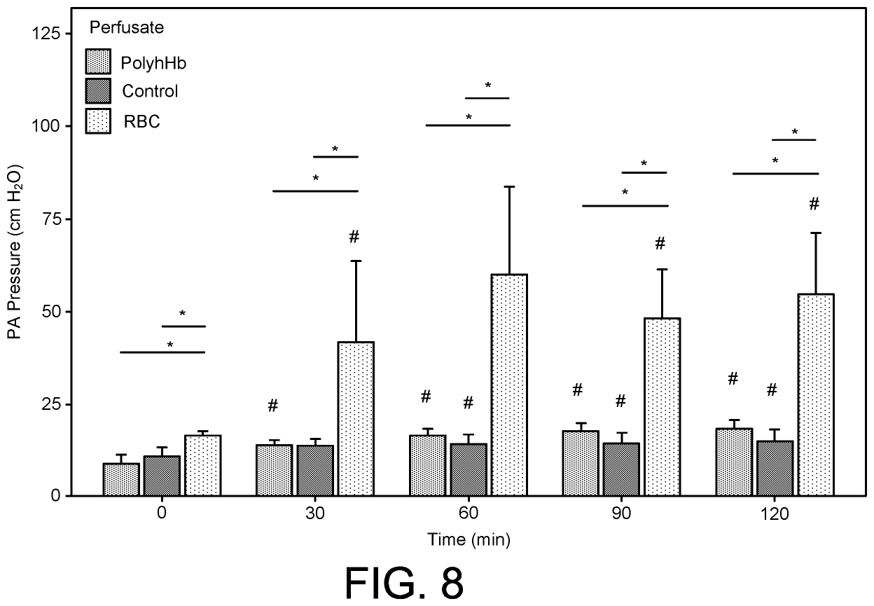

Beyond the ability to exchange gases, organ health on the circuit can be evaluated by measuring various physiological parameters during NEVLP. PA pressure is one of the most critical metrics of proper lung function, and a PA pressure elevated above 100 cm H₂O is a disqualifying event for continuation of NEVLP. All three perfusates led to an increase in PA pressure by 60 minutes as shown in FIG. 8. Lungs perfused using RBCs experienced higher PA pressures with three lungs being removed from the circuit after 60 minutes upon exceeding 100 cm H₂O. Returning to the discussion of FIG. 2, RBCs experienced significant hemolysis throughout NEVLP. Hemolysis leads to high levels of cytotoxic cell-free Hb in solution leading to, among other aforementioned side-effects, vasoconstriction. The vasoactive nature of cell-free Hb in conjunction with the fact that RBCs are lysing during NEVLP explains why the PA pressure of the RBC perfusate was much higher than the other two perfusates.

FIG. 8 shows that there is no significant difference in PA pressure between lungs perfused with PolyhHb and the asanguinous control. One of the shortcomings of previous generations of HBOCs is the presence of significant levels of low MW Hb species (<500 kDa), which elicited vasoconstriction and systemic hypertension, similar to what was observed for RBC perfusates. The lack of vasoactivity in this PolyhHb perfusate not only bodes well for the health of the organ on the circuit, but also confirms the lack of cytotoxicity due to the absence of cell-free Hb and low MW Hb species (<500 kDa) in the present PolyhHb.

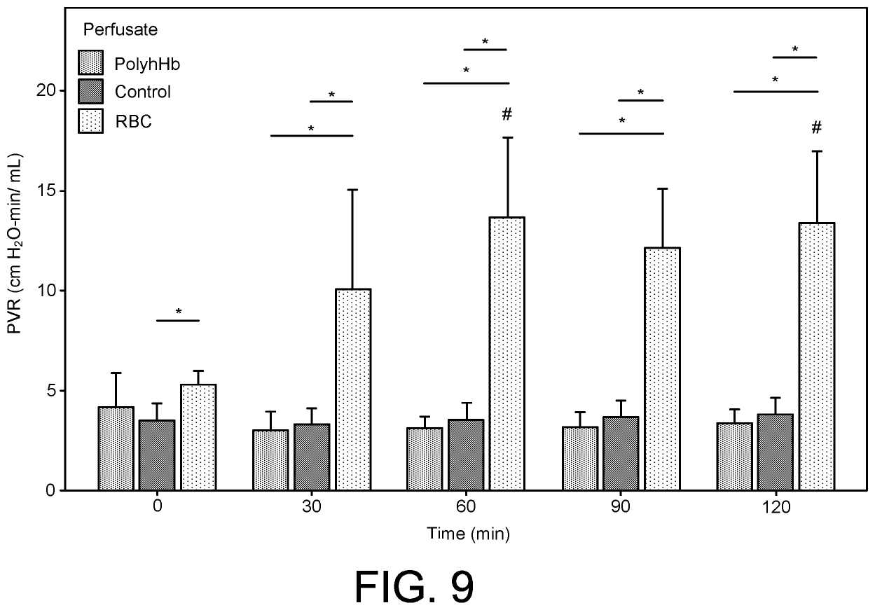

The trends displayed by the PA pressure over the course of NEVLP were mimicked by the results of tracking the pulmonary vascular resistance (PVR). FIG. 9 shows the PVR as a function of time and is similar to the results observed for the PA pressure. In both cases, the lungs perfused with RBCs exhibited prohibitively higher physiological responses compared to lungs perfused with both PolyhHb and the colloid control. Additionally, RBC perfused lungs were the only group to increase significantly from the t=0 point. These results are likely also due to the vasoactive nature of cell-free Hb derived from lysed RBCs compared to the asanguinous control or PolyhHb solution optimized to have such low MW Hb species (<500 kDa) removed from solution.

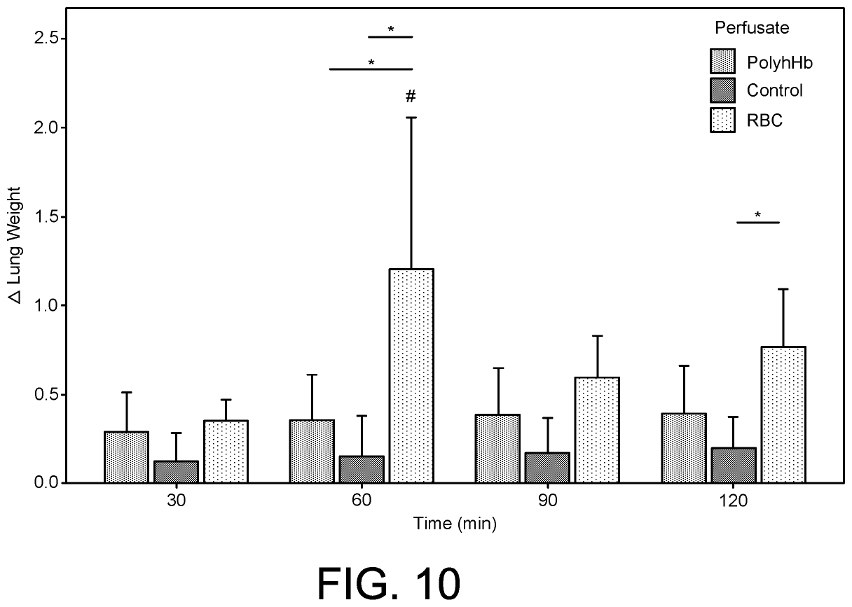

Along with vasoconstriction and systemic hypertension, the cytotoxicity caused by cell-free Hb and low MW Hb polymers (<500 kDa) is another serious side-effect associated with previous generations of PolyhHbs. On the NEVLP circuit, this manifests in real time in the change in lung weight. There are significant increases in lung weight when tissue is damaged leading to edema. The change in lung weight over time for the three perfusates is shown in FIG. 10. For RBCs, the lung weight spikes at t=60 due to the three failed lungs which were then taken off the circuit. Lungs perfused with PolyhHb or the asanguinous control did not display a significant increase in lung weight.

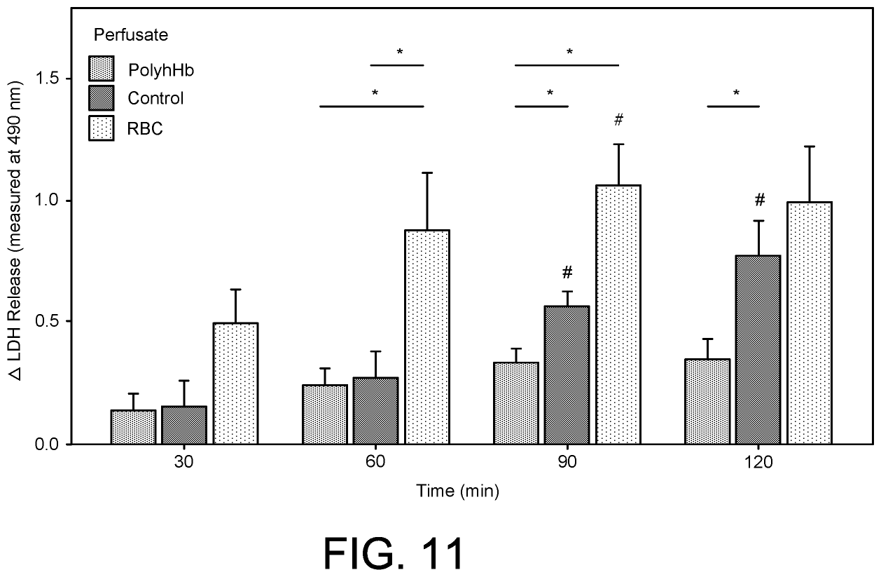

FIG. 11 shows the change in LDH during NEVLP. The change in perfusate LDH levels are lower for the PolyhHb perfusate compared to RBCs at 60 min and compared to both perfusates by 90 min. Additionally, the PolyhHb perfusate is the only group to not exhibit a significant increase in LDH compared to the t=30 min point. This means that performing NEVLPs using PolyhHb as a perfusate elicits less cellular stress and cell death compared to either RBC or asanguinous perfusates. Furthermore, the edema accumulated during NEVLP and quantified by the wet to dry ratio was shown to be significantly lower in lungs perfused with PolyhHb. The ratio of the right inferior lobe weight immediately after perfusion to the weight after 48 hours of drying is shown in FIG. 12. PolyhHb perfused lungs accumulated significantly less edema than lungs perfused with either RBCs or the asanguinous control. While the measurements made during NEVLP showed the advantages of using PolyhHb as a perfusate, the results from post-NEVLP analyses shown in FIGS. 11 and 12 show improvements in graft health by using the PolyhHb perfusate.

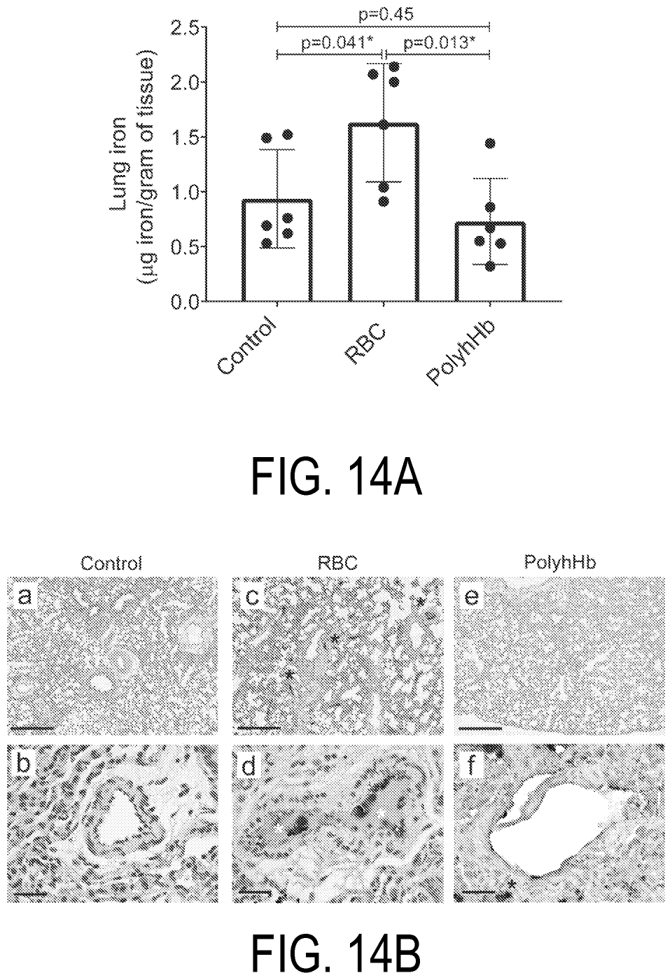

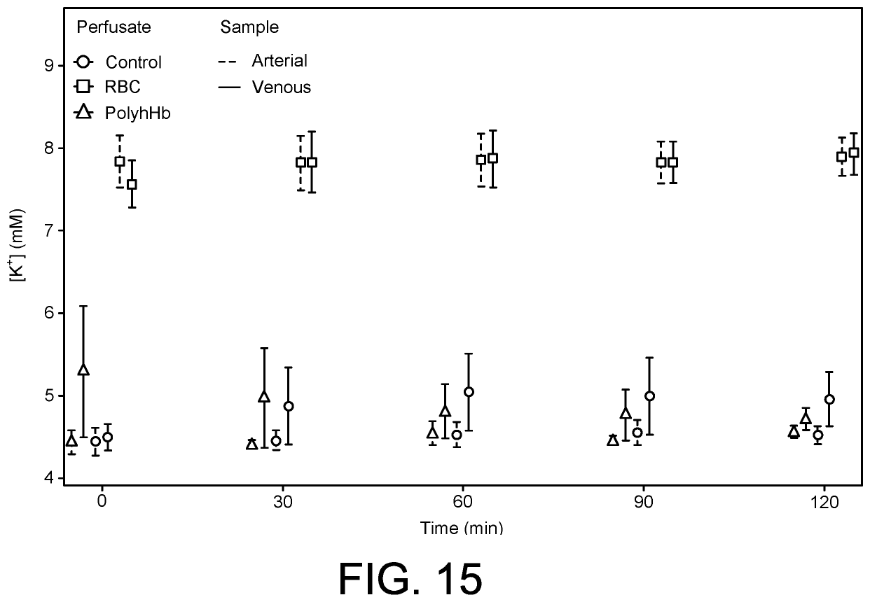

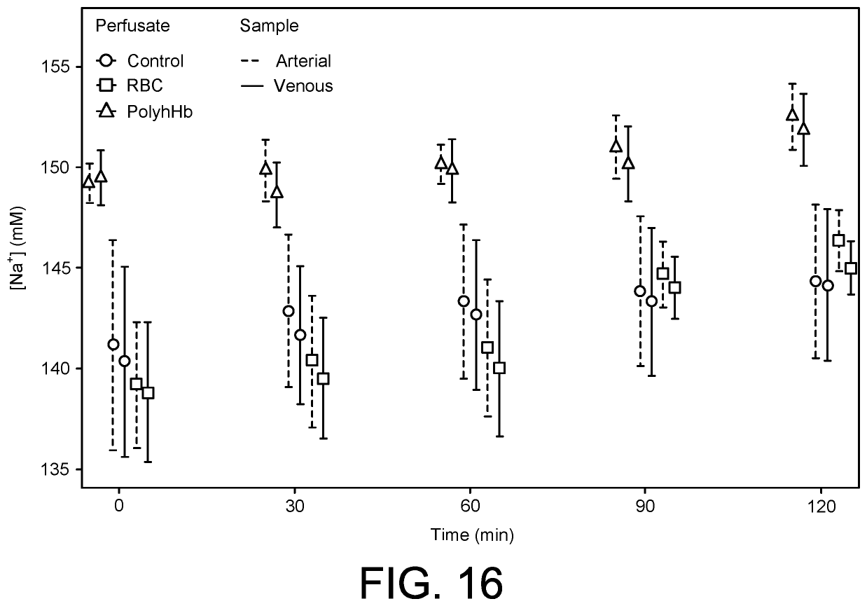

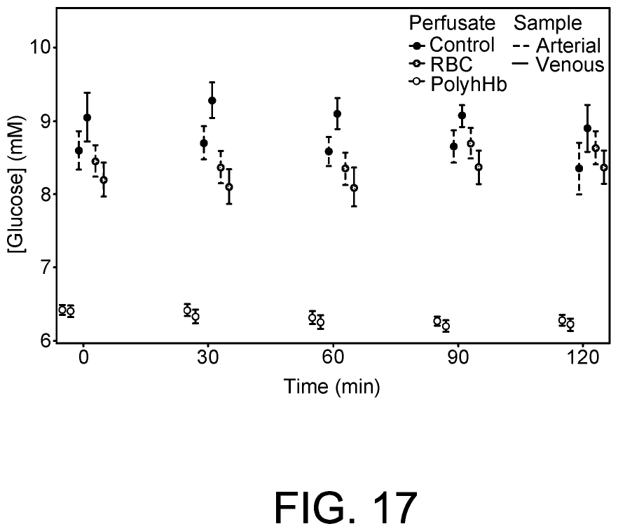

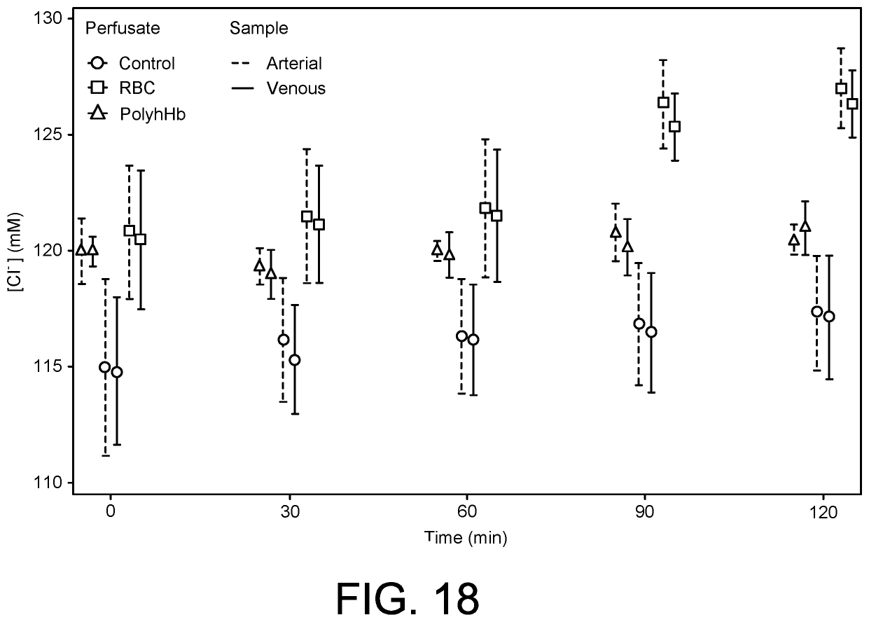

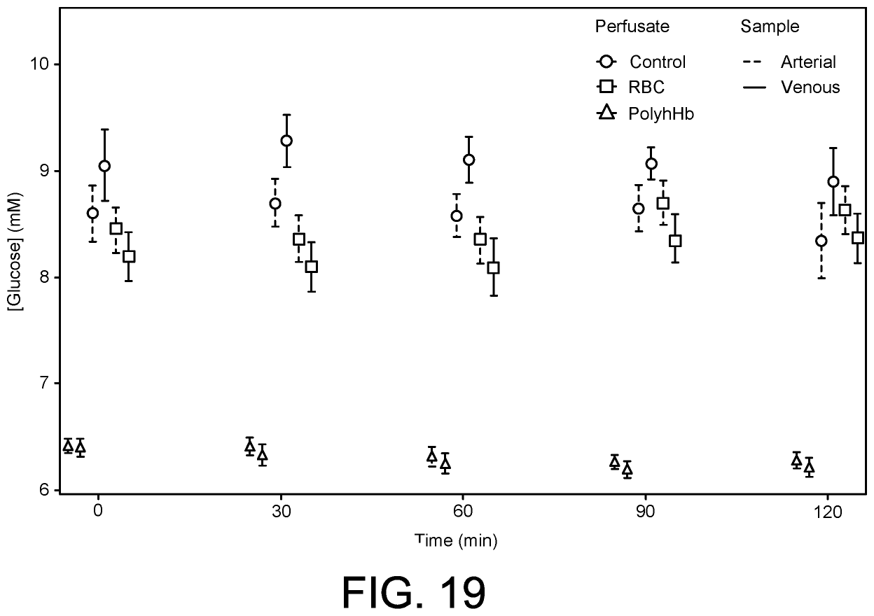

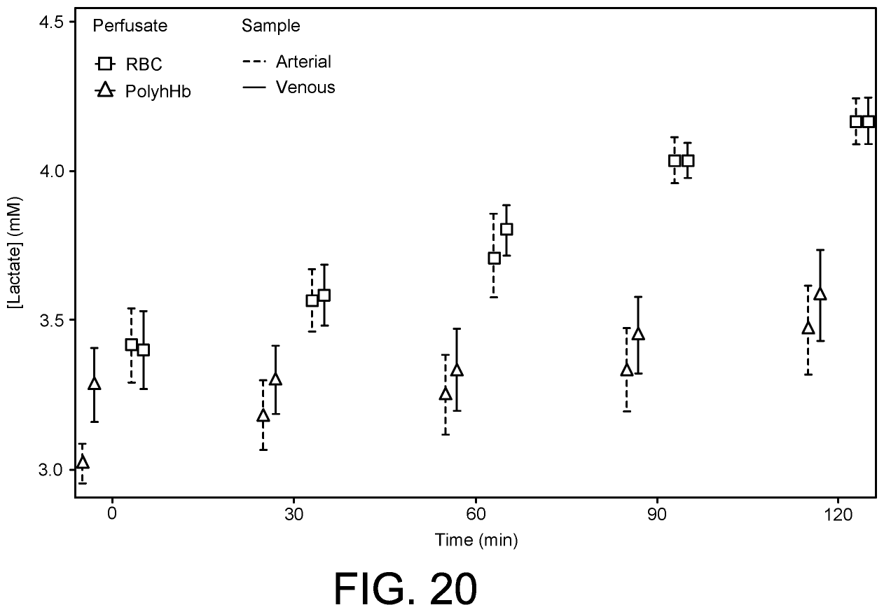

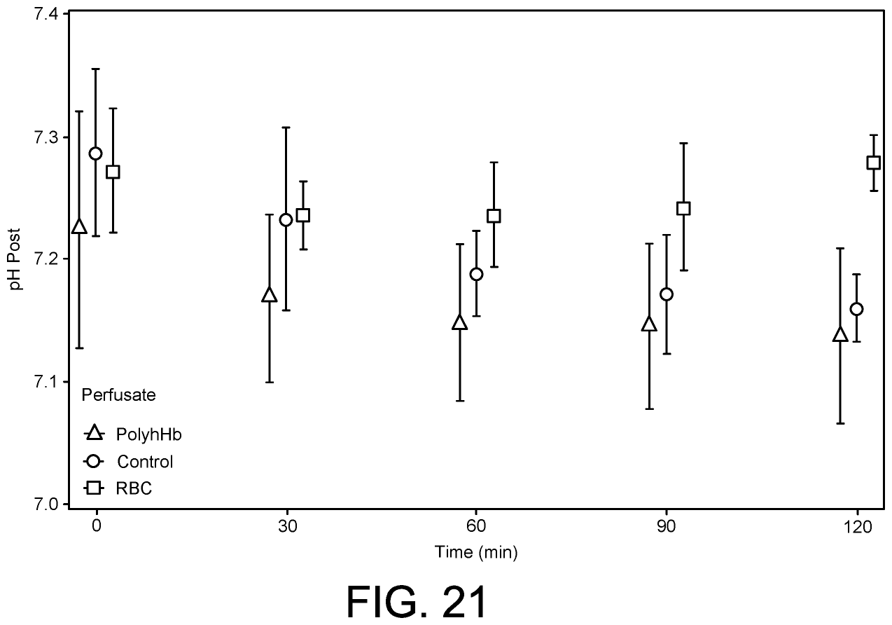

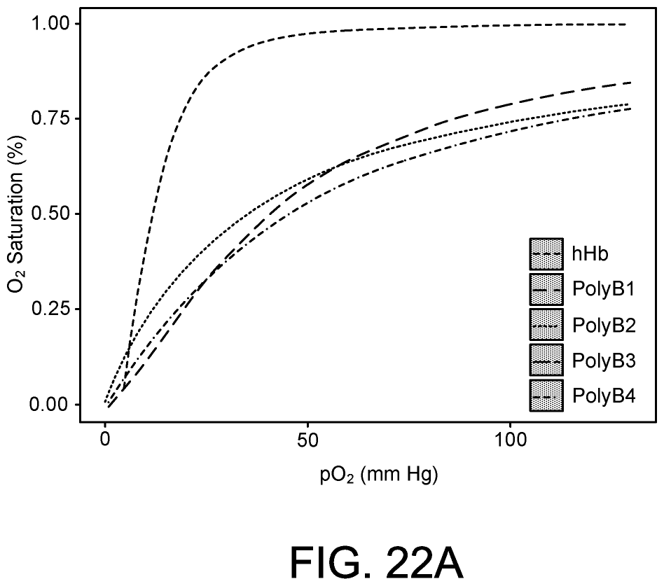

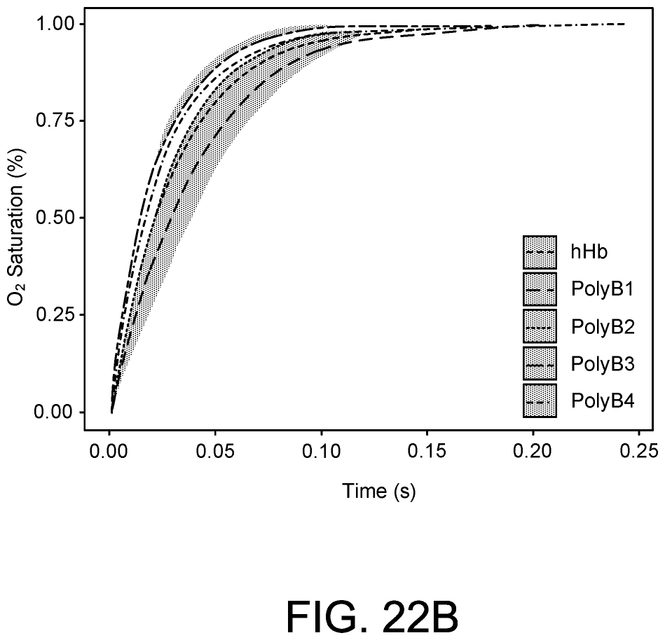

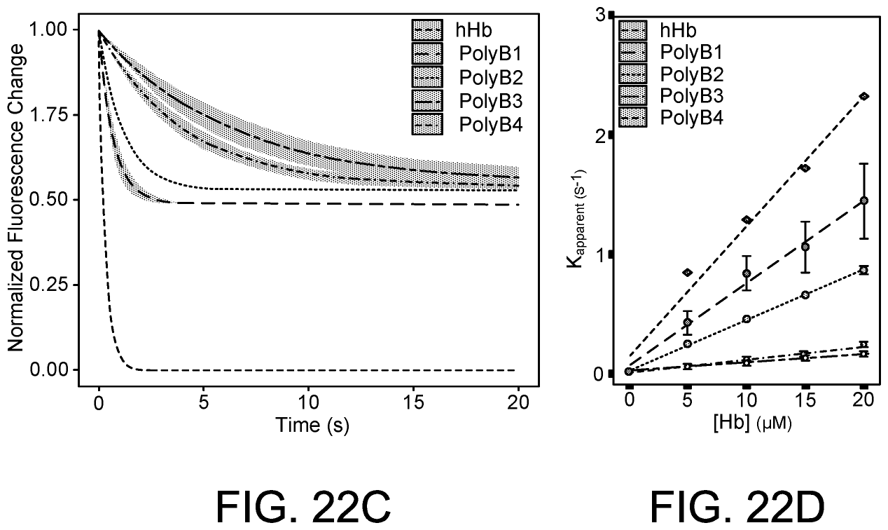

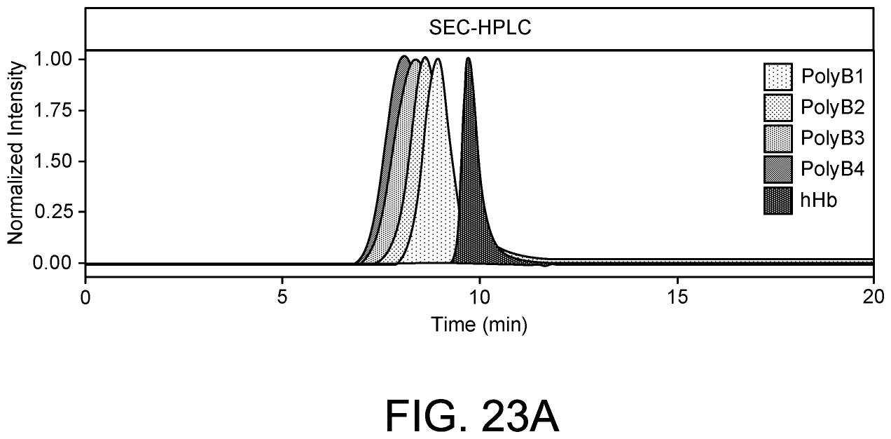

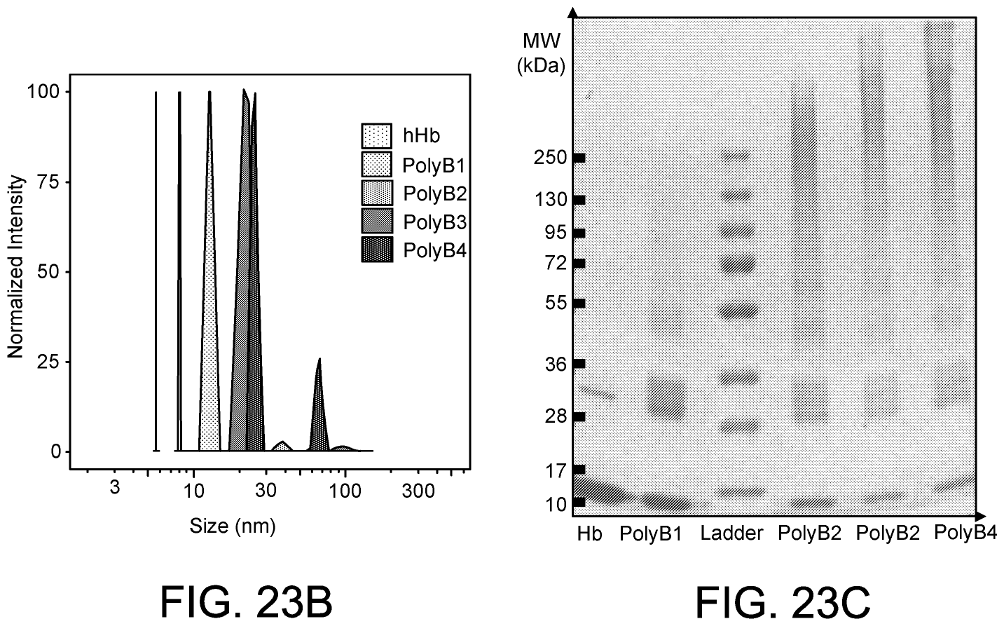

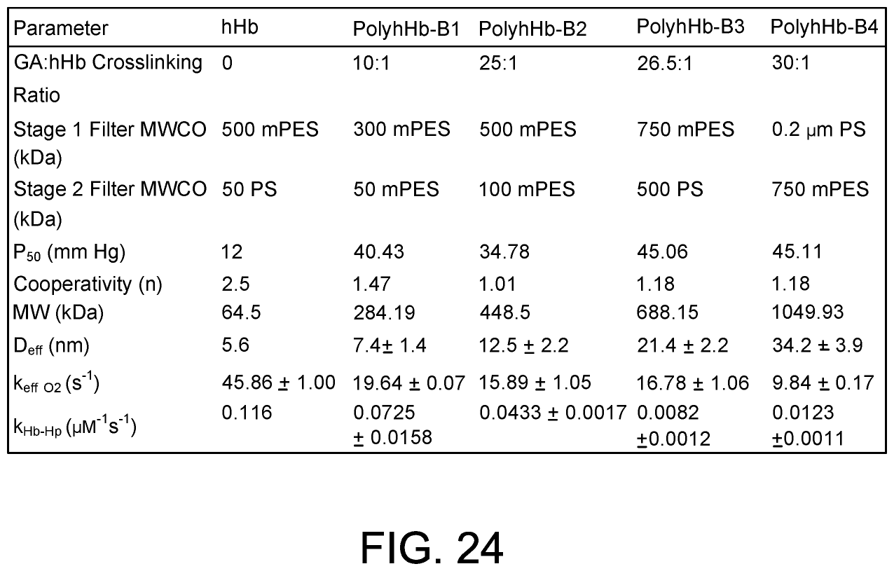

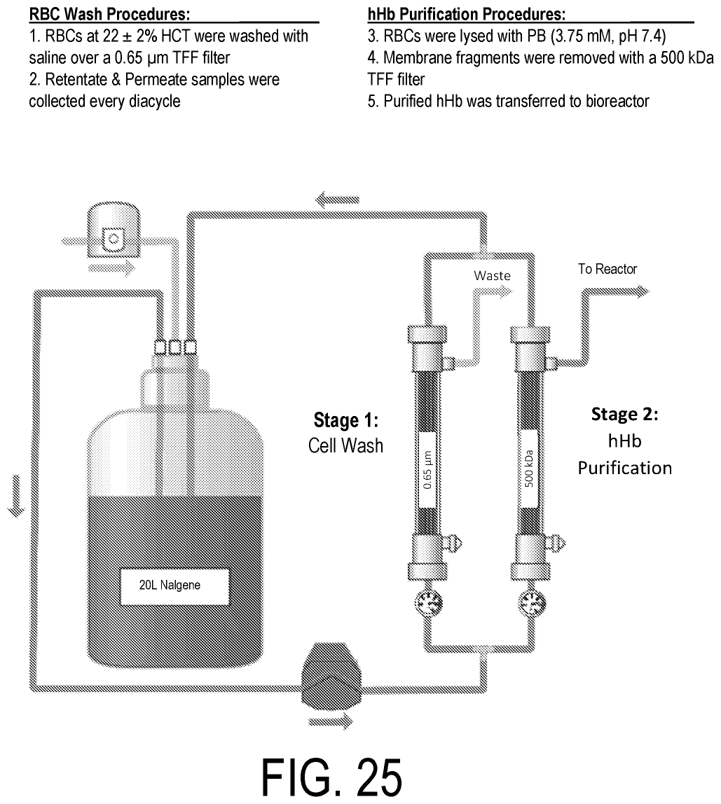

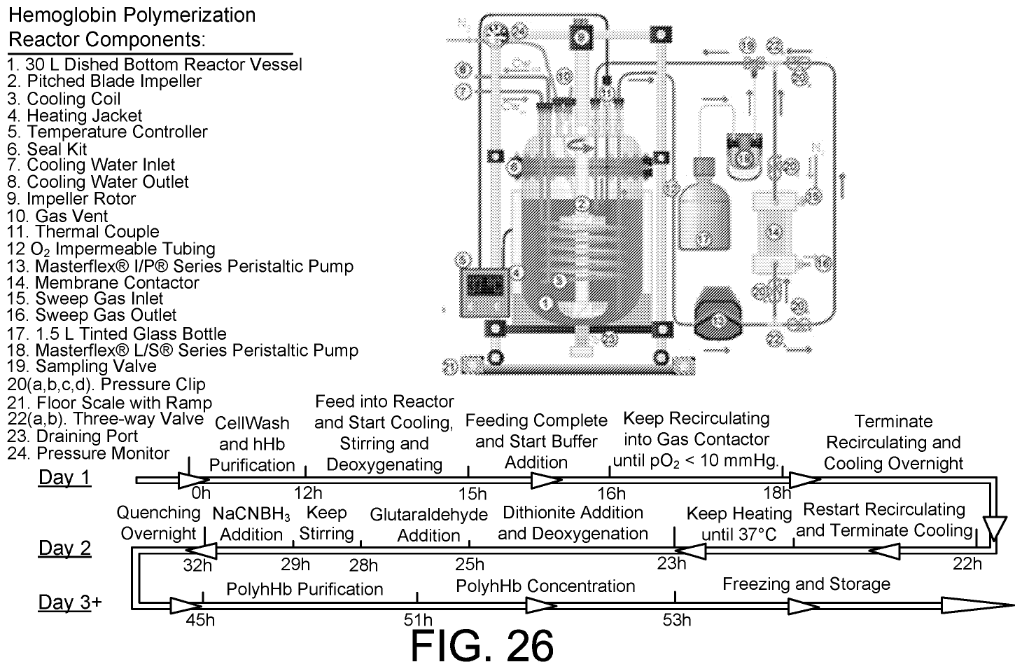

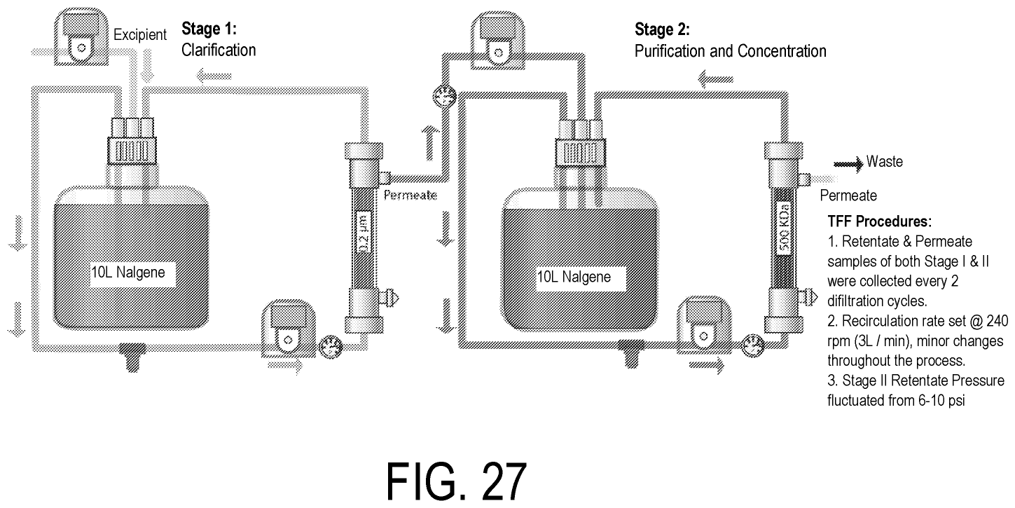

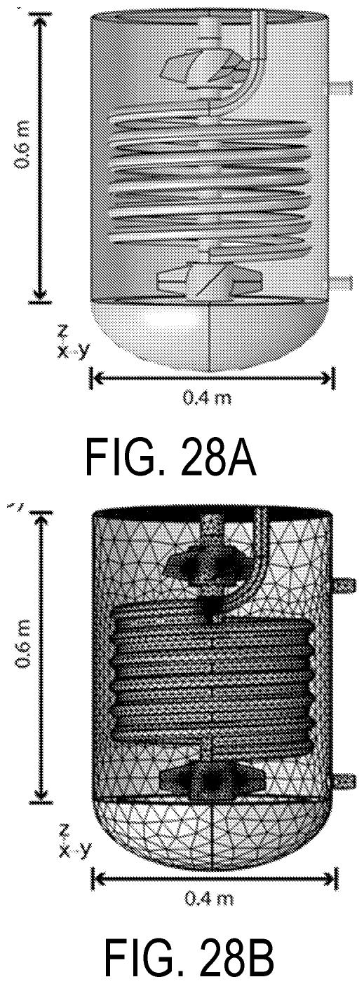

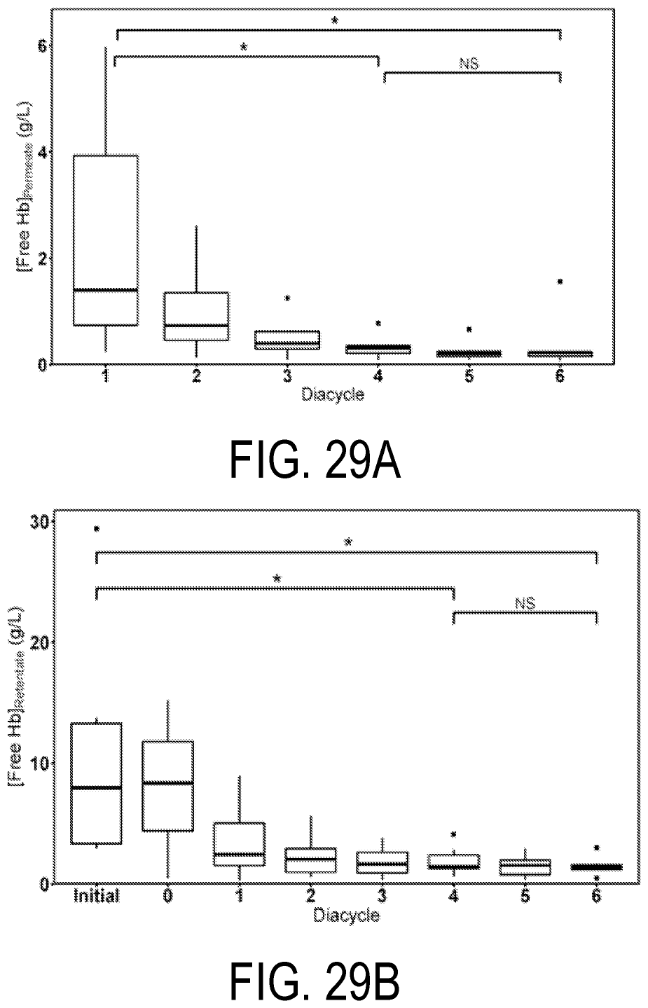

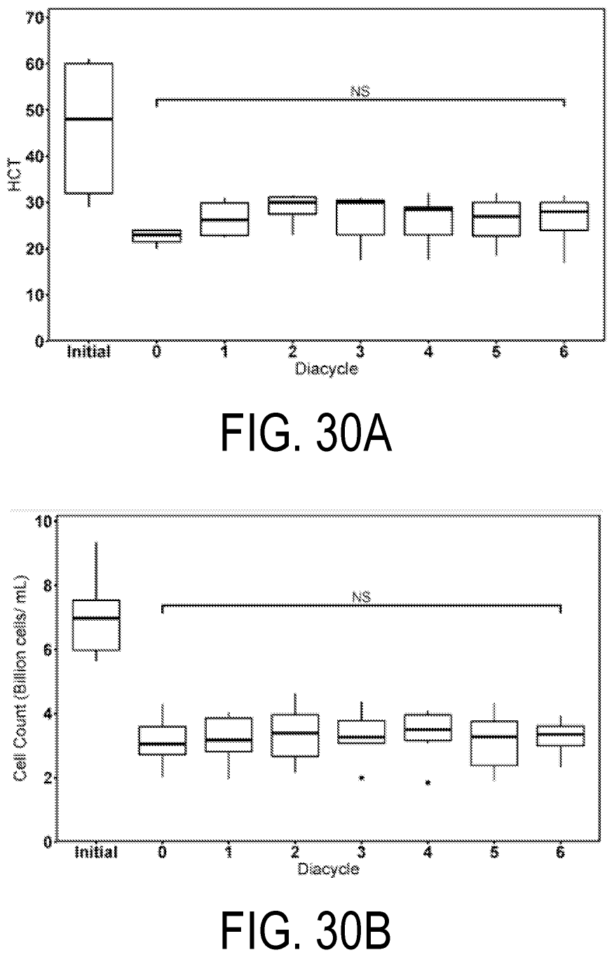

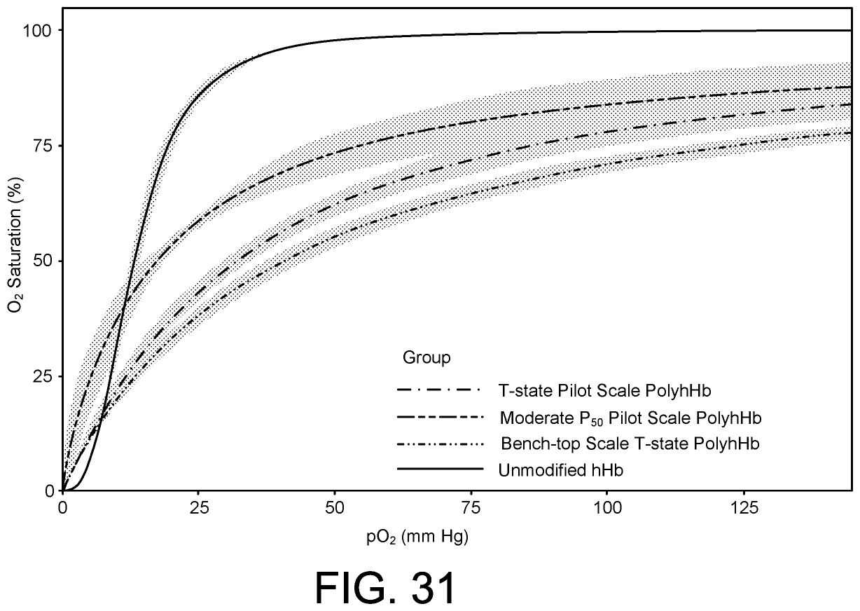

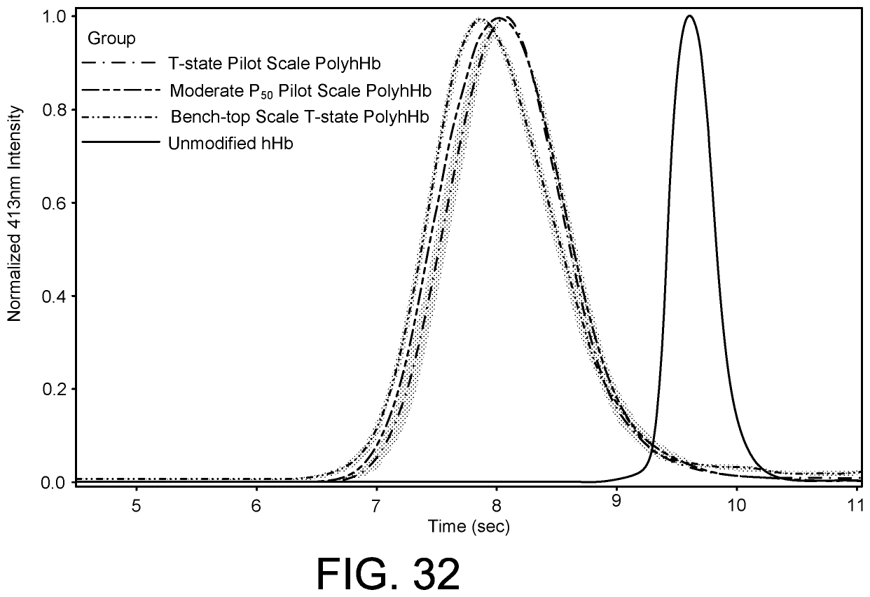

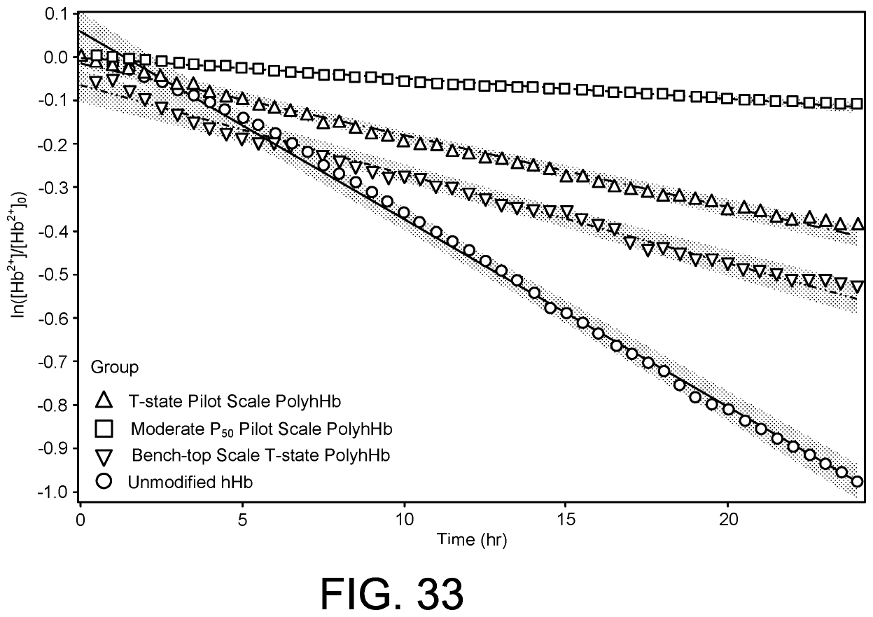

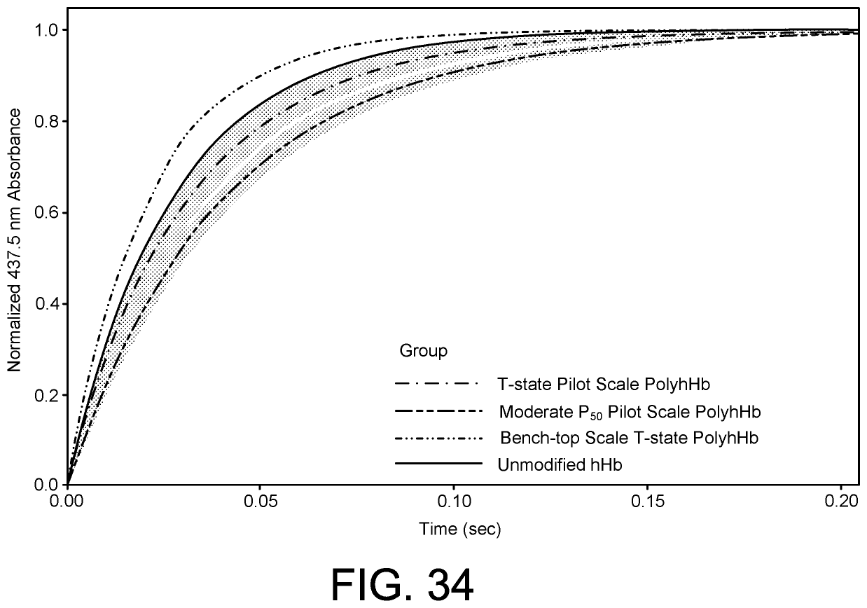

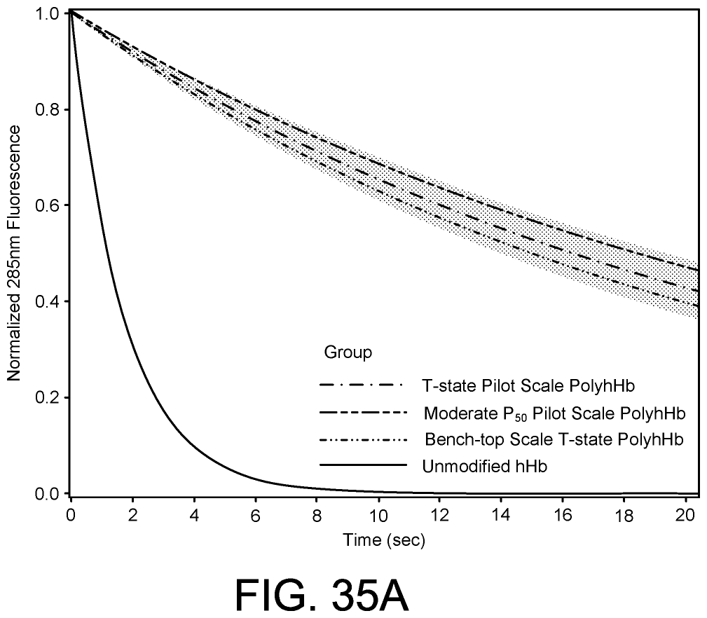

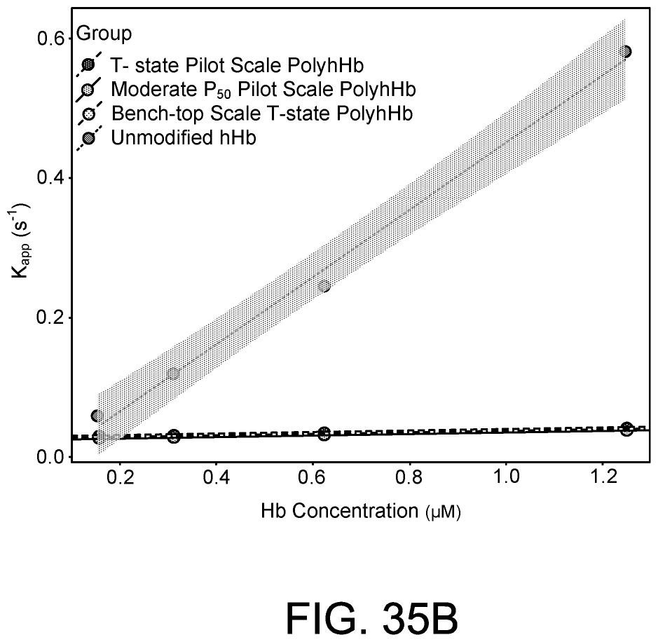

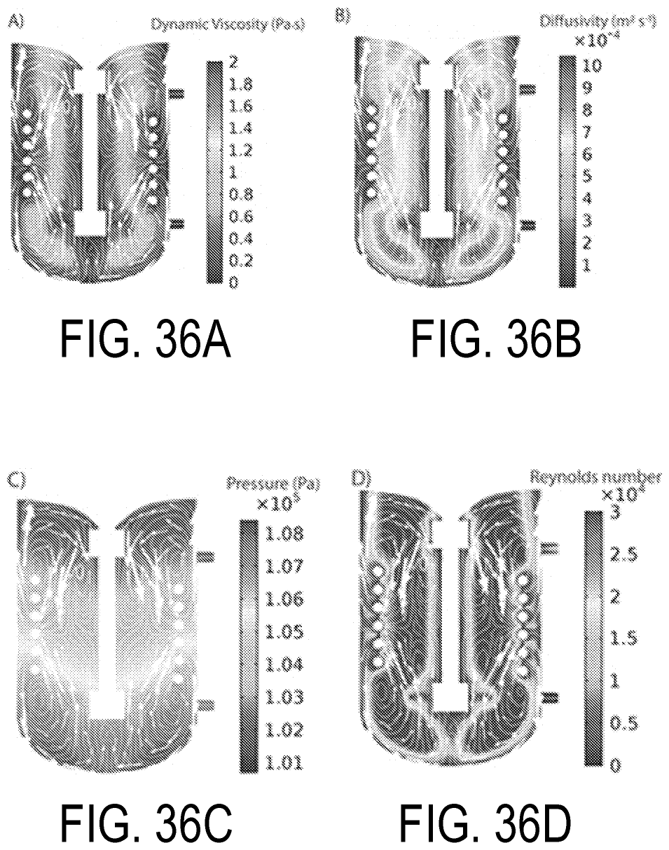

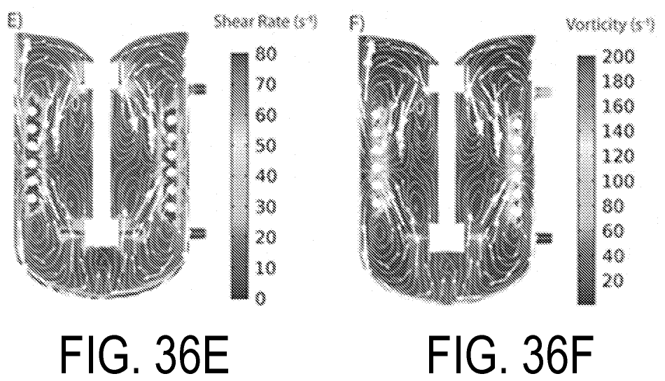

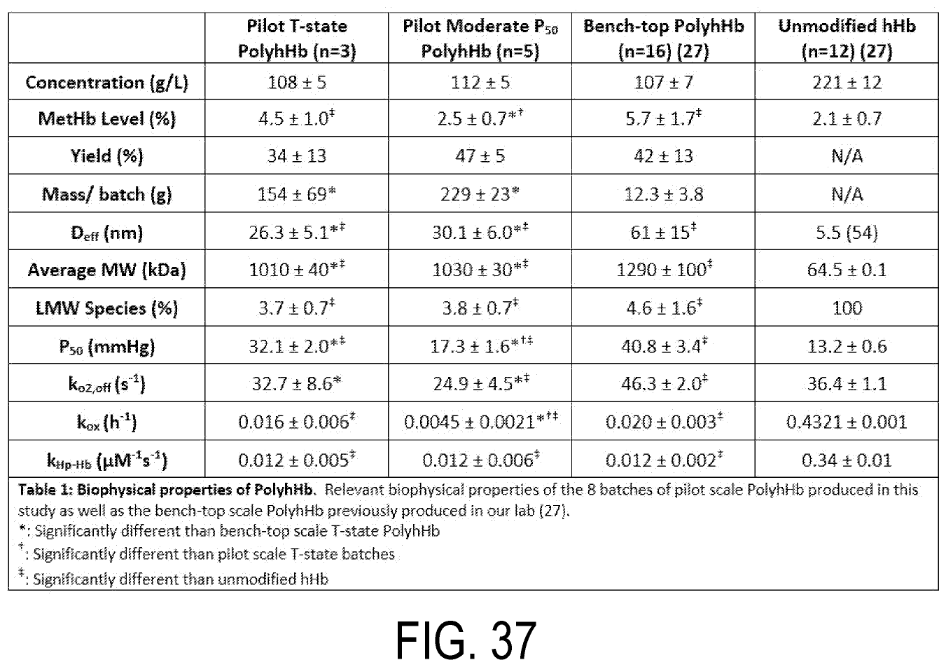

Lung tissue iron was quantified as a parameter of Hb degradation following perfusion of explanted tissue. RBC perfused tissue retained visually greater quantities of Hb compared to the control or PolyhHb perfused lungs prior to tissue homogenization and ferrozine assay analysis. Ferrozine functions as a water-soluble Fe²⁺ chelator with an absorbance at 562 nm and is therefore specific to reaction with unconjugated iron. Based on this data, RBC lung perfusion resulted in an average iron concentration equal to 1.63 μg/g of lung tissue. RBC perfused tissue contained residual iron concentrations that were 74% greater than after control perfusion and 123% greater than after PolyhHb perfusion as shown in FIG. 14A.

### CONCLUSION

Traditionally, NEVLPs have been performed using either a colloid based asanguinous solution or an RBC-based perfusate. Both of these options have shortcomings in preserving graft health and viability during NEVLP. Earlier HBOCs including previous generations of PolyhHbs have caused detrimental side-effects due to the presence of cytotoxic cell-free Hb and other low MW Hb polymers in solution. Improvements to the synthesis and purification of PolyhHb described in this example yield a product that is significantly less likely to elicit the negative side effects observed in previous generations of PolyhHbs. Our PolyhHb demonstrates improved lung oxygenation as well as overall graft health by eliciting less edema, extravasation, iron deposition, and cellular damage. This improved HBOC is a perfusate for NEVLP, which delivers O₂ while simultaneously not damaging the lungs.

Other advantages which are obvious, and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

1. A method of preserving a biological tissue sample ex vivo, the method comprising:
contacting the tissue sample ex vivo with a perfusion solution comprising polymerized hemoglobin,
wherein the perfusion solution comprises less than 5% by weight low molecular weight hemoglobin species, based on the total weight of the perfusion solution.

2. The method of claim 1, wherein the low molecular weight hemoglobin species has a molecular weight below 300 kDa.

3. The method of claim 1, wherein the polymerized hemoglobin is prepared by a process that comprises:
polymerizing hemoglobin; and
filtering the perfusion solution by ultrafiltration against a filtration membrane having a pore size that separates the low molecular weight hemoglobin species from the polymerized hemoglobin.

4. The method of claim 3, wherein the filtration membrane is rated for retaining solutes having a molecular weight greater than a molecular weight of the low molecular weight species but less than a molecular weight of the polymerized hemoglobin, thereby forming a retentate fraction comprising the polymerized hemoglobin and a permeate fraction comprising the low molecular weight hemoglobin species.

5. (canceled)

6. The method of claim 4, wherein the retentate fraction comprises the polymerized hemoglobin having a molecular weight of greater than 300 kDa and the permeate fraction comprises the low molecular weight hemoglobin species having a molecular weight of less than 300 kDa.

7. The method of claim 6, wherein the polymerized hemoglobin is prepared by a process that further comprises filtering the retentate fraction comprising the polymerized hemoglobin by ultrafiltration against a second filtration membrane, thereby forming a second retentate fraction comprising the polymerized hemoglobin with a molecular weight above a cutoff value and a second permeate fraction comprising species having a molecular weight below the cutoff value and above 300 kDa.

8-12. (canceled)

13. The method of claim 7, wherein the polymerized hemoglobin is prepared by a process that further comprises filtering the second retentate fraction comprising the polymerized hemoglobin by ultrafiltration against a third filtration membrane, thereby forming a third retentate fraction comprising high molecular weight impurities with a molecular weight above a second cutoff value and a third permeate fraction comprising the polymerized hemoglobin with a molecular weight below the second cutoff value and above the cutoff value.

14-18. (canceled)

19. The method of claim 3, wherein the filtration membrane is rated for retaining solutes having a molecular weight greater than 0.2 μm, thereby forming a retentate fraction comprising species having a molecular weight of greater than 0.2 μm and a permeate fraction comprising the polymerized hemoglobin having a molecular weight of less than 0.2 μm and the low molecular weight hemoglobin species.

20. (canceled)

21. The method of claim 1, wherein the polymerized hemoglobin is prepared by a process that further comprises filtering the permeate fraction comprising the polymerized hemoglobin and the low molecular weight hemoglobin species by ultrafiltration against a second filtration membrane, thereby forming a second retentate fraction comprising the polymerized hemoglobin having a molecular weight below 0.2 μm and above a cutoff value and a second permeate fraction comprising the low molecular weight hemoglobin species having a molecular weight below the cutoff value.

22-26. (canceled)

27. The method of claim 1, wherein the tissue sample comprises an organ.

28-32. (canceled)

33. The method of claim 1, wherein the perfusion solution comprises 3-4 g/dL of the polymerized hemoglobin, 25-85 mM NaCl, 1-3 mM KCl, 6-20 mM KH₂PO₄, 20-70 mM sodium gluconate, 5-21 mM sodium lactate, 1-4 mM magnesium gluconate, 0.6-1.2 mM CaCl₂) dihydrate, 11-16 mM NaOH, 1-4 mM adenine, 2-8 mM dextrose, 0.5-3 mM glutathione, 2-8 mM HEPES, 1-4 mM ribose, 7-30 mM mannitol, 10-40 g/L hydroxyethyl starch, 40-160 mg/dL N-acetyl-L-cysteine, or any combination thereof.

34. (canceled)

35. The method of claim 1, wherein the method further comprises perfusing the organ prior to transplantation, perfusing the organ during surgery or treatment, or perfusing the organ prior to or during collection of cells from the organ.

36. The method of claim 1, further comprising maintaining the tissue at a temperature of from 18° C. to 37° C., such as from 20° C. to 37° C., of 25° C. to 37° C., or from 35° C. to 37° C.

37. (canceled)

38. The method of claim 1, where the tissue sample has ischemic damage.

39. The method of claim 1, wherein the perfusion solution comprises from 1% to 5% by weight albumin, based on total weight of the perfusion solution.

40. The method of claim 1, wherein the perfusion solution has an osmolarity from 270 to 370 mOsm, such as an osmolarity from 324 to 346 mOsm;
a viscosity from 2 cP to 4.5 cP at normothermic conditions, such as a viscosity from 2.9 to 3.7 cP at normothermic conditions;
a colloid osmotic pressure from 14 mm Hg to 20 mm Hg, such as a colloid osmotic pressure from 16.8 to 17.6 mm Hg;
or a combination thereof.

41. (canceled)

42. (canceled)

43. The method of claim 1, wherein the polymerized hemoglobin is synthesized at a molar ratio from 20:1 to 40:1 of glutaraldehyde and hemoglobin.

44. (canceled)

45. The method of claim 1, wherein the partial pressure of oxygen at which 50% of the polymerized hemoglobin is saturated with oxygen is from 1 mm Hg to 50 mm Hg, such as from 14 mm Hg to 16 mm Hg.

46. The method of claim 1, wherein the polymerized hemoglobin exhibits an oxidation rate is from 0.0020 to 0.0085 h⁻¹, such as from 0.0045 to 0.0065 h⁻¹.

47. A method of enhancing the performance of a composition comprising red blood cells in tissue storage or perfusion, the method comprising adding a perfusion solution described herein to the oxygen carrier comprising the red blood cells; wherein the polymerized hemoglobin improves the ability of the composition to oxygenate the tissue.
